(19) United States
(12) Patent Application Publication
(10) Pub. No.: US 2013/0209515 A1
(43) Pub. Date: Aug. 15, 2013

(54) ANTI-INFECTIVE AND IMMUNOMODULATORY COMPOUNDS

(71) Applicants: Laurence Rahme, Jamaica Plain, MA (US); François Lepine, Longueuil (CA); Eric Deziel, Mascouche (CA)

(72) Inventors: Laurence Rahme, Jamaica Plain, MA (US); François Lepine, Longueuil (CA); Eric Deziel, Mascouche (CA)

(21) Appl. No.: 13/596,680

(22) Filed: Aug. 28, 2012

Related U.S. Application Data
(63) Continuation of application No. 11/916,379, filed on Oct. 28, 2008, now abandoned.
(60) Provisional application No. 60/686,816, filed on Jun. 2, 2005.

(51) Int. Cl. C07C 229/56

(52) U.S. Cl. CPC C07C 229/56
USPC 424/278.1

(57) ABSTRACT

The present invention provides pharmaceutical compositions and methods that include the use of anti-infective compounds that potentiate the host-immune response or limit or prevent the expression or activity of individual virulence factors. In addition, the compositions have immunomodulatory activity, and therefore can be used to prime host defenses to prevent or limit bacterial, fungal, and viral viability. In the compositions and methods of the inventions, specific steps of the bacterial-, fungal-, or viral-host interaction are targeted to prevent pathogenesis (e.g., infection). Such an approach should prevent pathogenic organisms from acquiring resistance to the protective anti-infective compounds.

### BACKGROUND OF THE INVENTION

This application relates to pharmaceutical compositions and methods that potentiate host-immune response to a pathogen or attenuate or prevent the expression or activity of pathogenic virulence factors.

In nature, most bacteria live not as individual cells but as pseudomulticellular organisms that coordinate their population behavior by means of small extracellular signal molecules. Under appropriate conditions, these molecules are released into the environment and taken up and responded to by surrounding cells (see Fuqua et al., Annu. Rev. Genet. 35:439-468, 2001; Miller and Bassler, Annu. Rev. Microbial. 55:165-199, 2001; Withers et al., Curr. Opin. Microbial. 4:186-193, 2001). “Quorum sensing” (QS), is the archetypal intercellular communication system used by many bacterial species to regulate their gene expression in response to cell density. This regulation allows all of the individual cells to behave coordinately and synergistically as a community, for instance, in growth dynamics and resource utilization (Fuqua et al., J. Bacterial. 176:269-275, 1994). A common feature of all QS systems is the transcriptional activation and repression of a large regulon of QS-controlled genes when a minimal threshold concentration of a specific autoinducer is reached.

The well-characterized QS system used by Gram-negative bacteria is mediated by N-acyl-L-homoserine lactones (AHLs) as extracellular signaling molecules (Fuqua et al., Annu. Rev. Genet. 35:439-468, 2001; Withers et al., Curr. Opin. Microbial. 4:186-193, 2001). The versatile and ubiquitous opportunistic pathogen Pseudomonas aeruginosa is one of the best-studied models of AHL-mediated QS. In this species, two separate autoinducer synthase/transcriptional regulator pairs, LasRI and RhlRI, modulate the expression of several genes, including many virulence factors, in response to increasing concentrations of the specific signaling molecules oxo-C₁₂-HSL and C₄-HSL (Pesci and Iglewski, Cell-Cell Signaling in Bacteria, eds. Dunny, G. M. & Winans, S. C. (Am. Soc. Microbiol., Washington, D.C.), pp. 147-155, 1999; Van Delden and Iglewski Emerg. Infect. Dis. 4:551-560, 1998).

P. aeruginosa also produces a cell-to-cell signal distinct from AHLs: 3,4-dihydroxy-2-heptylquinoline, called PQS (Pesci et al., Proc. Natl. Acad. Sci. USA 96:11229-11234, 1999). PQS serves as a signaling molecule regulating the expression of a subset of genes belonging to the QS regulon, including the phz and hcn operons. PQS functions in the QS hierarchy by linking a regulatory cascade between the las and the rhl systems (McKnight et al., J. Bacteriol. 182:2702-2708, 2000). That maximal PQS production occurs at the end of the exponential growth phase (Lépine et al., Biochim. Biophys. Acta 1622:36-40, 2003) supports the hypothesis that PQS acts as a secondary regulatory signal for a subset of QS-controlled genes. Although PQS has no antibiotic activity, it belongs to a family of poorly characterized antimicrobial P. aeruginosa products, the “pyo” compounds, originally described in 1945, which are derivatives of 4-hydroxy-2-alkylquinolines (HAQs) (Hays et al. J. Biol. Chem. 159:725-750, 1945; Wells, J. Biol. Chem. 196:331-340, 1952). A QS-associated P. aeruginosa transcriptional regulator, MvfR. (multiple virulence factor Regulator), which is required for the production of several secreted compounds, including virulence factors, and PQS, has also been identified (Cao et al., Proc. Natl. Acad. Sci. USA 98:14613-14618, 2001; Rahme et al., Proc. Natl. Acad. Sci. USA 94:13245-13250, 1997). Indeed, MvfR controls the synthesis of 2-aminobenzoic acid, a PQS precursor (Calfee et al., Proc. Natl. Acad. Sci. USA 98:11633-11637, 2001), by positively regulating the transcription of phnAB, which encodes an anthranilate synthase (Cao, vide supra). In addition, mutations in four genes, designated pqsABCD, result in loss of pyocyanin and PQS production (Gallagher et al., J. Bacteriol. 184:6472-6480, 2002; D'Argenio et al., J. Bacteriol. 184:6481-6489, 2002). These genes mediate HAQ synthesis (Déziel et al., Proc. Natl. Acad. Sci. USA 101:1339-1344, 2004).

Furthermore, via genome-wide expression studies using the Affymetrix GeneChip P. aeruginosa oligonucleotide array, it has been demonstrated that the MvfR transcriptional regulator controls pqsA-E expression (Déziel et al., vide supra). These results revealed the HAQ biosynthesis pathway and showed that one HAQ congener, 4-hydroxy-2-heptylquinoline (HHQ), is the direct precursor of PQS, and is itself a message molecule involved in cell-to-cell communication. This pathway represents a target for the pharmacological intervention of infections by organisms that utilize a quorum sensing mechanism, such as, for example, P. aeruginosa-mediated infections.

### SUMMARY OF THE INVENTION

Presented herein are results demonstrating that halogenated 2-aminobenzoic acid compounds, 2′-aminoacetophenone, and analogs thereof, are involved in the 4-hydroxy-2-alkylquinoline (HAQ) pathway and have anti-infective and/or immunomodulatory properties when administered to mice before exposure to infection.

Accordingly, in a first aspect, the invention features a pharmaceutical composition that includes a pharmaceutically acceptable excipient and a compound having the formula:

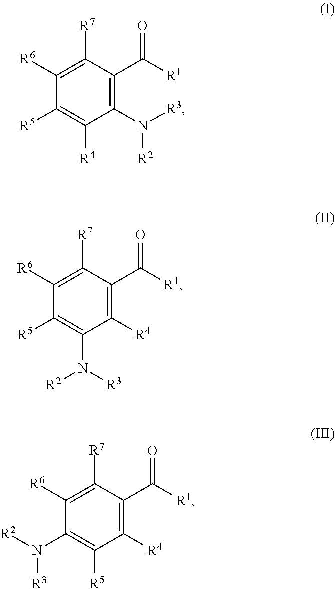

or a pharmaceutically acceptable salt or prodrug thereof. Desirably, the composition includes a compound of formula I.

In a compound of formula I, II, or III, R¹ is optionally substituted C₁₋₁₂ alkyl, optionally substituted C₃₋₈ cycloalkyl, optionally substituted C₂₋₁₂ alkenyl, optionally substituted C₂₋₁₂ alkynyl, optionally substituted C₁₋₄ alkaryl, or optionally substituted C₁₋₄ alkheterocyclyl; each of R² and R³ is, independently, H, optionally substituted C₁₋₆ alkyl, optionally substituted C₁₋₄ alkaryl, or optionally substituted C₁₋₄ alkheterocyclyl, or R², R³, and the nitrogen to which they are bonded together form a nitro group; R⁴ is H, Hal, OH, or C₁₋₆ alkoxy; and each of R⁵, R⁶, or R⁷ is, independently, H, OH, Hal, optionally substituted C₁₋₆ alkyl, or optionally substituted C₁₋₆ alkoxy. In an embodiment, each of R² and R³ is H. Examples of a compound of formula I include 2′-aminoacetophenone and 2′-amino-3-hydroxyacetophenone.

In another aspect, the invention features a pharmaceutical composition comprising a pharmaceutically acceptable excipient and a compound of having formula:

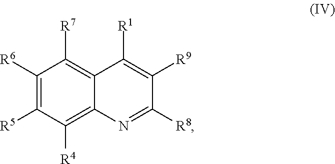

or a pharmaceutically acceptable salt or prodrug thereof. Each of the R¹, R⁴, R⁵, R⁶, and R⁷ groups is as defined above for a compound of formula I. Additionally, in a compound of formula IV, R⁸ is optionally substituted C₁₋₁₂ alkyl, optionally substituted C₃₋₈ cycloalkyl, optionally substituted C₂₋₁₂ alkenyl, optionally substituted C₂₋₁₂ alkynyl, optionally substituted C₁₋₄ alkaryl, or optionally substituted C₁₋₄ alkheterocyclyl and R⁹ is H, OH, optionally substituted C₁₋₆ alkoxy, optionally substituted C₁₋₁₂ alkyl, optionally substituted C₁₋₄ alkaryl, or optionally substituted C₁₋₄ alkheterocyclyl. In desirable examples, R¹ is C₁₋₄ alkyl; each of R⁴, R⁵, R⁶, R⁷, and R⁹ is H; and R⁸ is C₅₋₁₂ alkyl.

In another aspect, the invention features a pharmaceutical composition comprising a pharmaceutically acceptable excipient and compound 1, compound 2, compound 3, compound 4, or compound 5, with these compounds having the following structures:

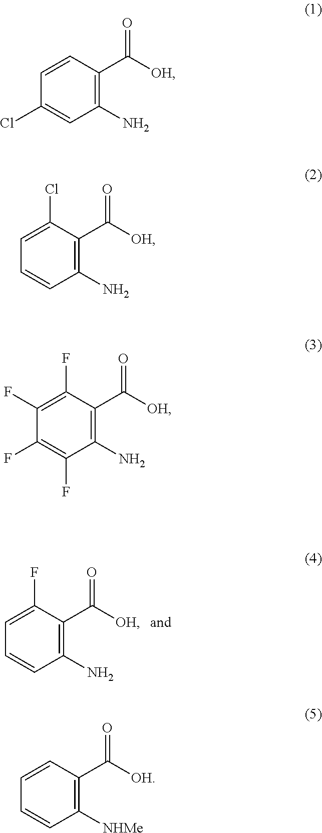 [SMILES: Nc1cc(Cl)ccc1C(=O)O ; Nc1cccc(Cl)c1C(=O)O ; Nc1c(F)c(F)c(F)c(F)c1C(=O)O ; Nc1cccc(F)c1C(=O)O ; CNc1ccccc1C(=O)O]

In another aspect, the invention features a method for treating a microbial infection, e.g., a bacterial infection, fungal infection, or viral infection, in an animal that includes administering to the animal an effective amount a compound of formula I, formula II, formula III, or formula IV, or any of compounds 1 to 5.

In yet another aspect, the invention features a method for enhancing the innate immune response for mitigating the effects or propagation of a disease, such as, for example, bacterial infection, fungal infection, viral infection, autoimmune disease, allergic condition, or cancer in an asymptomatic animal that includes administering to the animal an effective amount a compound of formula I, formula II, formula III, or formula IV, or any of compounds 1 to 5.

In an embodiment of any of the methods of the invention, the microbial infection is the result of a pathogenic bacterial infection, fungal infection, or viral infection. Examples of pathogenic bacteria include, without limitation, Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia. Specific examples of such bacteria include Vibrio harveyi, Vibrio cholerae, Vibrio parahaemolyticus, Vibrio alginolyticus, Pseudomonas phosphoreum, Pseudomonas aeruginosa Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes, or Staphylococcus aureus.

In another embodiment, the infection is the result of a Gram-negative bacterium.

### DEFINITIONS

The terms “acyl” or “alkanoyl,” as used interchangeably herein, represent an alkyl group, as defined herein, or hydrogen attached to the parent molecular group through a carbonyl group, as defined herein, and is exemplified by formyl, acetyl, propionyl, butanoyl and the like. Exemplary unsubstituted acyl groups include from 2 to 7 carbons.

The terms “C_{x-y} alkaryl” or “C_{x-y} alkylenearyl,” as used herein, represent a chemical substituent of formula —RR′, where R is an alkyl group of x to y carbons and R′ is an aryl group as defined elsewhere herein. Similarly, by the terms “C_{x-y} alkheteroaryl” “C_{x-y} alkyleneheteroaryl,” is meant a chemical substituent of formula RR″, where R is an alkyl group of x to y carbons and R″ is a heteroaryl group as defined elsewhere herein. Other groups preceeded by the prefix “alk-” or “alkylene-” are defined in the same manner.

The term “alkenyl,” as used herein, represents monovalent straight or branched chain groups of, unless otherwise specified, from 2 to 6 carbons containing one or more carbon-carbon double bonds and is exemplified by ethenyl, 1-propenyl, 2-propenyl, 2-methyl-1-propenyl, 1-butenyl, 2-butenyl, and the like.

The term “alkoxy” represents a chemical substituent of formula —OR, where R is an alkyl group of 1 to 6 carbons, unless otherwise specified.

The term “alkoxyalkyl” represents an alkyl group to which is attached an alkoxy group. Exemplary unsubstituted alkoxyalkyl groups include between 2 to 12 carbons.

The terms “alkyl” and the prefix “alk-,” as used herein, are inclusive of both straight chain and branched chain saturated groups of from 1 to 6 carbons, unless otherwise specified. Alkyl groups are exemplified by methyl, ethyl, n- and iso-propyl, n-, sec-, iso- and tert-butyl, neopentyl, and the like, and may be optionally substituted with one, two, three or, in the case of alkyl groups of two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) amino; (5) aryl; (6) arylalkoxy; (7) aryloyl; (8) azido; (9) carboxaldehyde; (10) cycloalkyl of three to eight carbon atoms; (11) halo; (12) heterocyclyl; (13) (heterocycle)oxy; (14) (heterocycle)oyl; (15) hydroxyl; (16) N-protected amino; (17) nitro; (18) oxo; (19) spiroalkyl of three to eight carbon atoms; (20) thioalkoxy of one to six carbon atoms; (21) thiol; (22) —CO₂R^{A}, where R^{A} is selected from the group consisting of (a) alkyl, (b) aryl and (c) alkaryl, where the alkylene group is of one to six carbon atoms; (23) —C(O)NR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (24) —SO₂R^{D}, where R^{D} is selected from the group consisting of (a) alkyl, (b) aryl and (c) alkaryl, where the alkylene group is of one to six carbon atoms; (25) —SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; and (26) —NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkynyl,” as used herein, represents monovalent straight or branched chain groups of from two to six carbon atoms containing a carbon-carbon triple bond and is exemplified by ethynyl, 1-propynyl, and the like.

The term “amino,” as used herein, represents an —NH₂ group.

The term “aminoalkyl,” as used herein, represents an alkyl group, as defined herein, substituted by an amino group.

The term “and/or” as used herein is meant to encompass alternative or inclusive combinations. For example, the statement “group A, group B, and/or group C” encompasses seven possibilities; each of the individual groups (3 possibilities), all of the groups together (1 possibility), and any two of the groups together (3 possibilities).

The term “aryl,” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like, and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) heteroaryl; (14) alkaryl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) alkaryl, where the alkylene group is of one to six carbon atoms; (37) —(CH₂)_{q}CONR^{B}R^{C}, where R^{B} and R^{C} are independently selected from the group consisting of (a) hydrogen, (b) alkyl, (c) aryl and (d) alkaryl, where the alkylene group is of one to six carbon atoms; (38) —(CH₂)_{q}SO₂R^{D}, where R^{D} is selected from the group consisting of (a) alkyl, (b) aryl and (c) alkaryl, where the alkylene group is of one to six carbon atoms; (39) —(CH₂)_{q}SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “cycloalkyl,” as used herein represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl groups of this invention can be optionally substituted with (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) alkaryl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) alkaryl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (38) —(CH₂)_{q}SO₂R^{D}, where R^{D} is selected from the group consisting of (a) alkyl, (b) aryl and (c) alkaryl, where the alkylene group is of one to six carbon atoms; (39) —(CH₂)_{q}SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term an “effective amount” or a “sufficient amount” of an agent, as used herein, is that amount sufficient to effect beneficial or desired results, such as clinical results, and, as such, an “effective amount” depends upon the context in which it is being applied. For example, in the context of administering an agent that is an anti-infective, an effective amount of the agent is, for example, an amount sufficient to achieve a reduction of microbial growth or dissemination as compared to the response obtained without administration of the agent.

The terms “halide” or “halogen” or “halo,” as used herein, represent bromine, chlorine, iodine, or fluorine.

The term “heteroaryl,” as used herein, represents that subset of heterocycles, as defined herein, which are aromatic: i.e., they contain 4n+2 pi electrons within the mono- or multicyclic ring system. Exemplary unsubstituted heteroaryl groups are of from 1 to 9 carbons.

The terms “heterocycle” or “heterocyclyl,” as used interchangeably herein represent a 5-, 6- or 7-membered ring, unless otherwise specified, containing one, two, three, or four heteroatoms independently selected from the group consisting of nitrogen, oxygen and sulfur. The 5-membered ring has zero to two double bonds and the 6- and 7-membered rings have zero to three double bonds. The term “heterocycle” also includes bicyclic, tricyclic and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring, such as indolyl, quinolyl, isoquinolyl, tetrahydroquinolyl, benzofuryl, benzothienyl and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidinyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazolyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

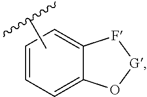

where

F′ is selected from the group consisting of —CH₂—, —CH₂O— and —O—, and G′ is selected from the group consisting of —C(O)— and —(C(R′)(R″))ᵥ—, where each of R′ and R″ is, independently, selected from the group consisting of hydrogen or alkyl of one to four carbon atoms, and v is one to three and includes groups, such as 1,3-benzodioxolyl, 1,4-benzodioxanyl, and the like. Any of the heterocycle groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) heteroaryl; (14) alkaryl, where the alkylene group is of one to six carbon atoms; (15) aryloyl; (16) azido; (17) azidoalkyl of one to six carbon atoms; (18) carboxaldehyde; (19) (carboxaldehyde)allyl, where the alkylene group is of one to six carbon atoms; (20) cycloalkyl of three to eight carbon atoms; (21) cycloalkylalkyl, where the cycloalkyl group is of three to eight carbon atoms and the alkylene group is of one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) alkaryl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (38) —(CH₂)_{q}SO₂R^{D}, where R^{D} is selected from the group consisting of (a) alkyl, (b) aryl and (c) alkaryl, where the alkylene group is of one to six carbon atoms; (39) —(CH₂)_{q}SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “hydroxy,” as used herein, represents an —OH group.

The terms “N-protecting group” or “nitrogen protecting group” as used herein, represent those groups intended to protect an amino group against undesirable reactions during synthetic procedures. Commonly used N-protecting groups are disclosed in Greene and Wuts, “Protective Groups In Organic Synthesis, 3^{rd} Edition” (John Wiley & Sons, New York, 1999), which is incorporated herein by reference. N-protecting groups comprise acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and chiral auxiliaries such as protected or unprotected D, L or D, L-amino acids such as alanine, leucine, phenylalanine and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl and the like, arylalkyl groups such as benzyl, triphenylmethyl, benzyloxymethyl and the like and silyl groups such as trimethylsilyl and the like. Preferred N-protecting groups are formyl, acetyl, benzoyl, pivaloyl, t-butylacetyl, alanyl, phenylsulfonyl, benzyl, t-butyloxycarbonyl (Boc) and benzyloxycarbonyl (Cbz).

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable prodrugs” as used herein, represents those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “Ph” as used herein means phenyl.

The term “prodrug,” as used herein, represents compounds which are rapidly transformed in vivo to the parent compound of the above formula, for example, by hydrolysis in blood. Prodrugs of the compounds of the invention may be conventional esters that are hydrolyzed to their active carboxylic acid form. Some common esters which have been utilized as prodrugs are phenyl esters, aliphatic (C₈-C₂₄) esters, acyloxymethyl esters, carbamates and amino acid esters. In another example, a compound of the invention that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

Asymmetric or chiral centers may exist in any of the compounds of the present invention. The present invention contemplates the various stereoisomers and mixtures thereof. Individual stereoisomers of compounds of the present invention are prepared synthetically from commercially available starting materials which contain asymmetric or chiral centers or by preparation of mixtures of enantiometic compounds followed by resolution well-known to those of ordinary skill in the art. These methods of resolution are exemplified by (1) attachment of a racemic mixture of enantiomers, designated (+/−), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R,” or “S,” depending on the configuration of substituents around the chiral carbon atom. Alternatively, enantiomers are designated as (+) or (−) depending on whether a solution of the enantiomer rotates the plane of polarized light clockwise or counterclockwise, respectively.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration, where the term “Z” represents substituents on the same side of the carbon-carbon double bond and the term “E” represents substituents on opposite sides of the carbon-carbon double bond. It is also recognized that for structures in which tautomeric forms are possible, the description of one tautomeric form is equivalent to the description of both, unless otherwise specified. For example, amidine structures of the formula —C(═NR^{Q})NHR^{T} and —C(NHR^{Q})═NR^{T}, where R^{T} and R^{Q} are different, are equivalent tautomeric structures and the description of one inherently includes the other.

It is understood that substituents and substitution patterns on the compounds of the invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

Other features and advantages of the present invention will become apparent from the following detailed description. It should be understood that the detailed description and the specific examples, while indicating preferred embodiments of the invention, are given by way of illustration only, and various changes and modifications within the spirit and scope of the invention will become apparent to those skilled in the art from this detailed description.

### BRIEF DESCRIPTION OF THE DRAWINGS

FIG. 1A is a graph showing 2AA production in PA14 cells, as measured by LC/MS, as a function of bacterial growth, as determined by measuring culture supernatant optical density.

FIG. 1B is a graph showing that 2AA is dramatically reduced in mvfR and pqsA mutant cells.

FIG. 2 is a graph showing the results of LC/MS HAQ quantification analysis of a Pseudomonas aeruginosa culture that has not been treated with 2′-aminoacetophenone and shows the levels of HAQs produced by wild type.

FIG. 3 is a graph showing the results of LC/MS HAQ quantification analysis of a Pseudomonas aeruginosa culture that has been treated with 2′-aminoacetophenone.

FIG. 4 is a graph showing the growth kinetics of Pseudomonas aeruginosa cultures in the presence and absence of 2′-aminoacetophenone (2AA), 3′-aminoacetophenone (3AA), 4′-aminoacetophenone (4AA), or 2′-nitroacetophenone (2NA).

FIG. 5A is a graph showing the results of LC/MS analysis of a Pseudomonas aeruginosa culture that has been treated with 4-chloro-2-aminobenzoic acid for HHQ, N-oxide, and PQS.

FIG. 5B is a graph showing the results of LC/MS analysis of a Pseudomonas aeruginosa culture that has been treated with 6-chloro-2-aminobenzoic acid for HHQ, N-oxide, and PQS.

FIG. 5C is a graph showing the results of LC/MS analysis of a Pseudomonas aeruginosa culture that has been treated with 6-fluoro-2-aminobenzoic acid for HHQ, N-oxide, and PQS.

FIG. 6 is a graph showing the growth kinetics of Pseudomonas aeruginosa cultures in the presence and absence of 4-fluoro-2-aminobenzoic acid, 5-fluoro-2-aminobenzoic acid, 6-fluoro-2-aminobenzoic acid, 4-chloro-2-aminobenzoic acid, and 6-chloro-2-aminobenzoic acid.

FIG. 7 is a graph showing that 2AA injection in the burn eschar protects mice from P. aeruginosa infection.

FIGS. 8A-8C are graphs showing that 2AA treatment protects burn and infected (BI) mice from PA14-induced mortality. FIG. 8A: IP injection of 2AA at 6 h, 2 days, and 3 days pre BI; FIG. 8B: IP injection of 2AA at 8 and 30 days; and FIG. 8C: IV injection of 2AA 4 days pre BI. h, hours; d, days

FIG. 9 is a chart showing the survival percentage of groups of mice pre-treated with 2′-aminoacetophenone (2AA), 3′-aminoacetophenone (3AA), 4′-aminoacetophenone (4AA), and 2′-nitroacetophenone (2NA) in a burn/infection animal model compared with an untreated control group. The treatment group was pre-treated four days prior to injury and infection.

FIG. 10 is a schematic showing the structure of 2AA and metabolites and analogs thereof.

FIGS. 11A and 11B are graphs showing survival curves for mice injected IV with 2AA or analogs/metabolites thereof 4 days prior to BI. FIG. 11A: Survival curves for mice injected IV with 2-amino-3-hydroxy-aminoacetophenone; and FIG. 11B: Survival curves for mice injected IV with three related 2AA analogs. Analogs and metabolites of 2AA provide less protection to mice than 2AA.

FIGS. 12A-12C are photomicrographs of lung tissue after 2AA treatment. FIG. 12A: Lungs 4 days post-2AA treatment. FIG. 12B: Lungs 48 hours post infection with PA14 (wild type). The lung parenchyma is infiltrated with inflammatory cells with large areas of consolidation. FIG. 12C: Lungs infected with PA14 4 days post-2AA treatment. Slight peribronchial infiltrate of inflammatory cells is present at 48 hours but no significant interstitial involvement. Magnification 10×.

FIG. 13 is a graph showing that the addition of 3 mM 2AA inhibits the production of HHQ and PQS.

FIG. 14 is a graph showing that PqsA-LacZ expression in PA14 is significantly reduced in the presence of 3 mM 2AA.

FIGS. 15A and 15B are graphs showing that PqsA-LacZ expression in response to HHQ (FIG. 15A) and PQS (FIG. 15B) is inhibited in the presence of 2AA.

FIG. 16 is a graph showing that PQS is not required for pqsA transcription in vivo. pqsA-lacZ β-galactosidase activity is fully activated in PA14 and in the pqsH isogenic mutant.

FIG. 17 is a graph showing the five-day survival curves for mice after burn injury and infection. Mice were inoculated with 10⁵ cells of P. aeruginosa PA14 and pqsH and mvfR mutants. N=15 for each strain from two independent experiments.

FIG. 18 is a graph showing LC/MS analysis of PQS and HHQ in PA14 and in the pqsH isogenic mutant.

FIG. 19 is a graph showing that HHQ and PQS are in vivo inducers of MvfR in P. aeruginosa. pqsA-lacZ β-galactosidase activity increases in the pqsH isogenic mutant in the presence of PQS or HHQ. Arrow indicates addition of HHQ and PQS at OD_{600 nm}=1.0. Activity is given in Miller units (MU), corrected or culture OD 600 nm.

FIGS. 20A and 20B show binding of MvfR to the pqsA promoter. FIG. 20A is a gel showing that MvfR binds to the pqsA promoter in the absence of HHQ or PQS, but that binding is increased in the presence of HHQ or PQS. A ³²P-labeled 174-bp DNA fragment containing the pqsA promoter region was mixed with E. coli cell lysate containing MvfR minus ligand (lanes 2-3); plus 40 pM HHQ (lanes 4-5); or plus 40 pM PQS (lanes 6-7). Protein added per reaction: lane 1, 0 ng/μl; lanes 2, 4, and 6, 30 ng/μl; and lanes 3, 5, and 7, 60 ng/μl. HHQ and PQS were added at the final concentration of 40 pM. Reaction mixtures were electrophoresed on 5% non-denatured polyacrylamide gels. FIG. 20B is a graph showing the densitometry of shifted bands.

FIG. 21 is a graph showing that the PqsA-D enzymes synthesize the signal for MvfR activation in E. coli in the presence of 10 mg/l AA.

FIG. 22 is a schematic showing the chemical structure of anthranilic acid and its analogs.

FIGS. 23A-23C are graphs showing the growth of PA14 in the presence of AA analogs. FIG. 23A: MS determination of HAQs production in PA14, and in PA14 in the presence of each inhibitor. FIG. 23B: Growth kinetics in response to 6-FABA, 6-CABA, or 4-CABA in the presence of 1.5 mM HHQ or PQS. FIG. 23C: MS determination of the concentration of 6-FABA, 6-CABA, or 4-CABA in LB.

FIG. 24 is a graph showing percent survival of BI mice following infection with PA14 and treatment with 6-FABA, 6CABA, or 4CABA. Thermally injured mice were infected with 5×10⁵ PA14 cells and injected IV 6 hrs post BI with 100 μl 20 mM AA analogs. Average of 3 experiments with n=10/experiment.

FIG. 25A is a graph showing production of HAQs by PA14 in the presence of 1.5 mM methylanthranilate.

FIG. 25B is a graph showing the growth kinetics of PA14 in the presence or absence of methylanthranilate.

FIG. 26 is a graph showing mice survival following BI and infection with PA14 in the presence or absence of 6-FABA. Thermally injured mice were infected with 5×10⁵ PA14 cells and injected IV post BI with 100 μl 120 mM 6-FABA at 6 hours or 12 hours, or at 6 hours and 24 hours. Average of 4 experiments with n=10/experiment.

FIGS. 27A-27C are graphs showing that treatment with 6FABA, 6CABA, and 4CABA limit bacterial systemic presence of P. aeruginosa in the underlying muscle (FIG. 27A), adjacent muscle (FIG. 27B), and blood (FIG. 27C) of the host. Set of animals were burned, infected with 5×10⁵ PA14 cells, and treated after 6 hours with 6FABA, 6CABA, or 4CABA, or injected with saline (control mice). The numbers above the dots represent the number of mice processed in each condition. The statistical significance of the difference in bacterial presence was measured by a Wilcoxon rank sum test. P values for the difference in adjacent muscle after 6FABA, 6CABA and 4CABA treatments were 0.00001, 0.00066, and 0.00267 respectively, and for the difference in blood after 6FABA, 6CABA and 4CABA treatments were 0.00147, 0.00331, and 0.00331 respectively.

FIGS. 28A and 28B are charts showing the concentration of PA14 in mice treated with 6-fluoro-2-aminobenzoic acid (6-FABA) 6 hrs post-burn and infection. The PA14 cfu/mg of tissue was determined at 12 hours and 24 hours post-burn and infection in muscle adjacent to the burn and infection (FIG. 28A) and in blood (FIG. 28B). FIG. 28A shows that 6-FABA limits significantly the systemic dissemination of P. aeruginosa. A dramatic effect is seen in the blood samples, as no bacteria are detected in the blood of treated mice at 24 hrs (FIG. 28B). These results correlate with the increased survival of the mice, as shown in FIGS. 29 and 30, for 6 hour and 12 hour post injury and infection treatments, respectively.

FIG. 29 is a graph showing survival of mice in a burn injury experiment, where mice were treated with 6-fluoro-2-aminobenzoic acid 6 hours after injury and infection. The ordinate of the graph is the percentage of survivors.

FIG. 30 is a graph showing survival of mice in a burn 12 hours after injury and infection. The ordinate of the graph is the percentage of survivors.

FIG. 31 is a graph showing that in P. aeruginosa anthranilic acid is also produced by the enzymatic systems PhnAB or TrpEG. The addition of 6-CABA, 6-FABA, or 4-CABA resulted in a transient accumulation of anthranilic acid; the control (PA14 only) did not show an accumulation of anthranilic acid. A pqsA mutant also showed a transient accumulation of anthranilic acid similar to that seen upon treatment of PA14 with 6-CABA, 6-FABA, or 4-CABA.

FIG. 32 is a graph showing that the addition of increasing concentrations of AA analogs in culture containing 1.5 mM of any of these inhibitors reversed the inhibition of PqsA enzymatic activity and lead to an increase in HAQ production, at least for 6-FABA and 6-CABA.

FIG. 33 is a schematic showing the synthesis of 2-alkyl-4H-3,1-benzoxazin-4-ones, which are structurally related to HAQs.

FIG. 34 is a schematic showing the functional categorization of mouse genes whose expression is significantly (p<0.05) up- or down-regulated by 2AA treatment at different time points. The Y axis represents the number of genes in each category.

FIG. 35 is a table listing immunity genes differentially expressed in response to 2AA

FIG. 36 is a schematic showing the pathways that are relevant to protection of mice due to 2AA treatment. The line designated by the arrow represents the threshold of 0.05 P-value, bars that cross the orange line are significantly changed by 2AA at different time points.

FIG. 37 is a schematic showing the components of the integrin signaling pathway that are changed at 96 hrs post 2AA injection. Excluding the structures designated as “ECM Protein,” “MLCP,” “Paxillin,” and “ROCK1,” the gray-colored structures designate the genes that are down regulated by 2AA. “ROCK1” is up regulated by 2AA. “ECM Protein,” “MLCP,” and “Paxillin” show no change in expression at 96 hrs.

FIG. 38 is a cluster graph showing the expression of differentially expressed genes in mice exposed to 2AA 4 days pre-BI. The colors represent the fold changes as compared to untreated mice. The analyses of the 2AA-treated BI samples surprisingly do not identify any up-regulated genes and only 23 down-regulated genes. These latter genes function in the inflammatory response that is highly induced by BI (left panel), and are dramatically reduced in the 2AA treated samples. Interestingly, these genes are either unchanged, or induced by 2AA alone (right panel).

### DETAILED DESCRIPTION OF THE INVENTION

The present invention is based on the recognition that i) bacterial-host interactions are mediated by large sets of bacterial virulence factors that mediate several discrete biological steps in an infected animal; and ii) these steps define distinct targets that can be modulated with intrinsic bacterial compounds, metabolites of these compounds, or related analogs. These compounds act as anti-infective agents to potentiate the host-immune response or limit or prevent the expression or activity of individual virulence factors, or even entire virulence gene networks. In addition, these compounds may have immunomodulatory activity, and therefore are used to prime host defenses to prevent or limit bacterial viability. A major advantage the approach of targeting specific steps of the bacterial-host interaction is that it should prevent pathogenic bacteria from acquiring resistance to the protective anti-infective compounds.

Accordingly, the present invention features a pharmaceutical composition comprising a pharmaceutically acceptable excipient and a compound of having formula

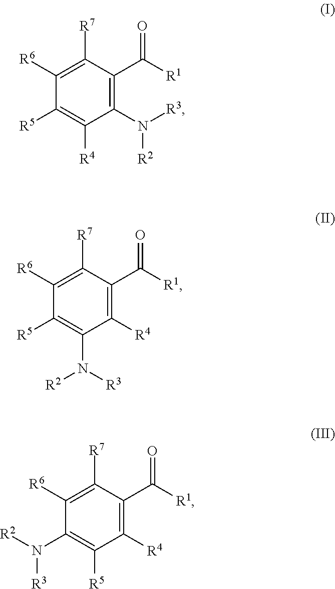

or where the various substituents are defined elsewhere herein. Examples of a compound of formula I include 2′-aminoacetophenone and 2′-amino-3-hydroxyacetophenone.

As it is presumed in the invention that 2′-aminoacetophenone and analogs may be metabolized in a manner similar to that proposed for the metabolism of 2-aminobenzoic acid, as shown in Scheme 1 (see Déziel et al., Proc. Natl. Acad. Sci. USA 101:1339-1344, 2004), the invention also features a compound of formula IV.

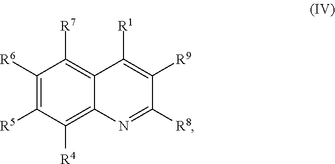

where the various substituents are as defined elsewhere herein. Examples of compounds of formula IV include those compounds where R¹ is C₁₋₄ alkyl, each of R⁴, R⁵, R⁶, R⁷, and R⁹ is H, and R⁸ is C₅₋₁₂ alkyl.

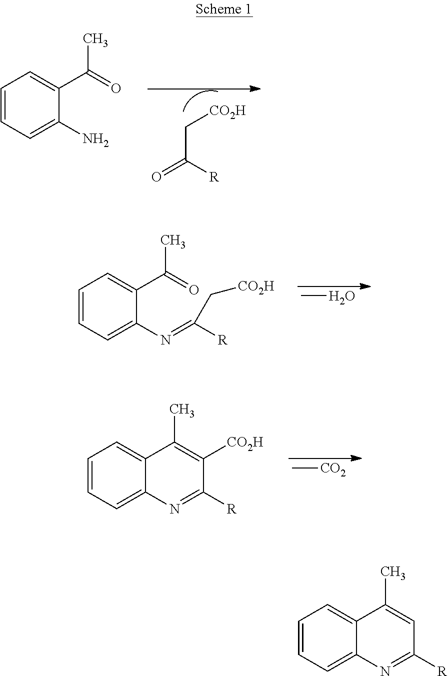

The invention also features a pharmaceutical composition comprising a pharmaceutically acceptable excipient and compound 1, compound 2, compound 3, compound 4, or compound 5, with these compounds having the following structures:

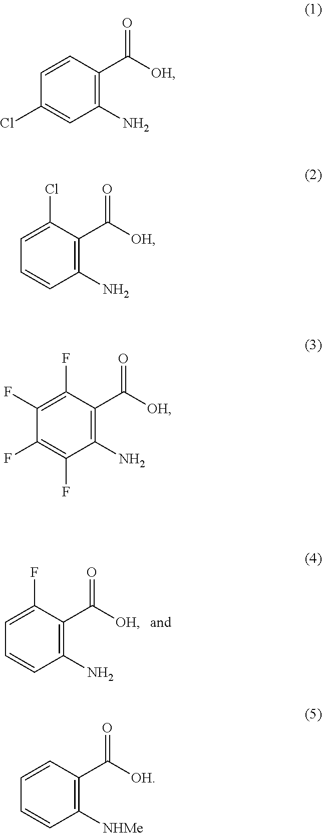 [SMILES: Nc1cc(Cl)ccc1C(=O)O ; Nc1cccc(Cl)c1C(=O)O ; Nc1c(F)c(F)c(F)c(F)c1C(=O)O ; Nc1cccc(F)c1C(=O)O ; CNc1ccccc1C(=O)O]

The compounds of formula I, II, III, or IV, or any of compounds 1 to 5, have anti-infective and/or immunomodulatory properties and are useful for the prophylaxis or treatment of pathological conditions such as bacterial infections, fungal infections, viral infections, autoimmune diseases, allergic conditions, or cancer.

For example, a composition of the invention can be used to modulate bacterial cell growth, bacterial virulence, siderophore expression, exopolysaccharide production in bacterial cells, bacteria colony morphology (including smooth colony morphology, such as that exhibited by a pathogenic bacterial cell), biofilm formation, and the like. In one embodiment, the biological activity results from any one of the following: Vibrio harveyi, Vibrio cholerae, Vibrio parahaemolyticus, Vibrio alginolyticus, Pseudomonas phosphoreum, Pseudomonas aeruginosa Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes, or Staphylococcus aureus.

### Preparation of Compounds Having Formulas I, II, III, and IV

A compound of formula I, where each of R¹, R², R³, R⁴, R⁵, R⁶, and R⁷ is as defined elsewhere herein, can be prepared, for example, as shown in Scheme 2. Accordingly, -2-aminobenzoic acid, or an analog thereof (e.g., a compound of formula V), is N-protected (e.g., by a Boc group) to produce a compound of formula VI, where P is the nitrogen protecting group. This compound is subsequently reacted with an alkoxyamine, such as, for example N-methoxymethylamine, under amide bond-forming conditions to form a Weinreb amide of formula VII. In one example, the amide bond-forming reaction can be mediated by a carbodiimide, such as, for example, diisopropylcarbodiimide. The Weinreb amide can be subsequently reacted with alkyl, alkenyl, alkynyl, alkaryl, or alkheterocyclyl carbanions, such as lithium salts or Grignard reagents, to form ketones of formula VIII, where R¹ is optionally substituted C₁₋₁₂ alkyl, optionally substituted C₂₋₁₂ alkenyl, optionally substituted C₂₋₁₂ alkynyl, optionally substituted C₁₋₄ alkaryl, or optionally substituted C₁₋₄ alkheterocyclyl, Removal of the amine protecting group forms a compound of formula IX. The amine of a compound of formula IX can be further elaborated to produce a compound of formula X or XI by methods well known to those skilled in the art, such as, for example, by reductive amination and/or by N-alkylation with a suitable alkylating agent (e.g., an alkyl halide).

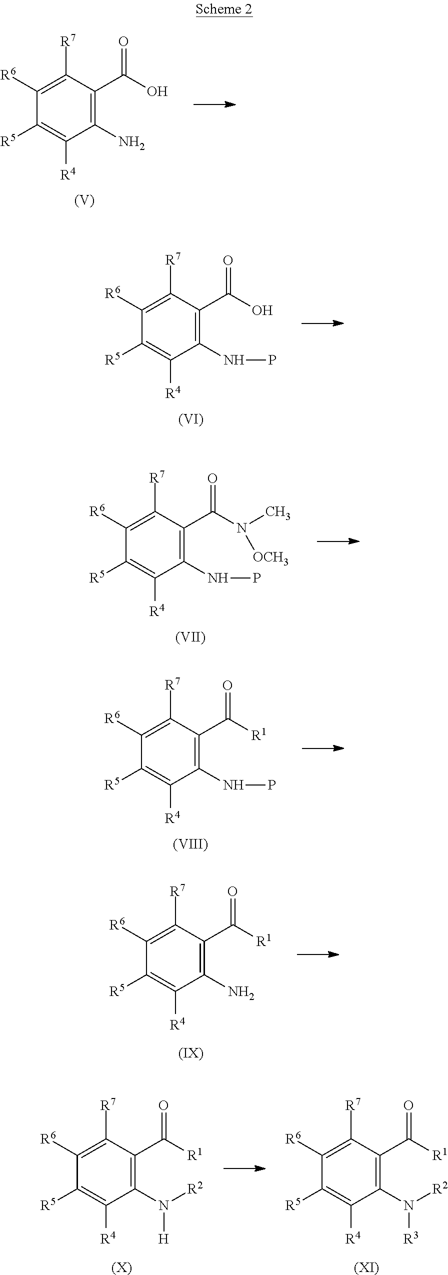

It should recognized by one skilled in the art that compounds of formulas II and III can be similarly prepared from compounds of formulas XII and XIII, respectively, as shown in Scheme 3, by the same methodology used to prepare a compound of formula XI from a compound of formula V.

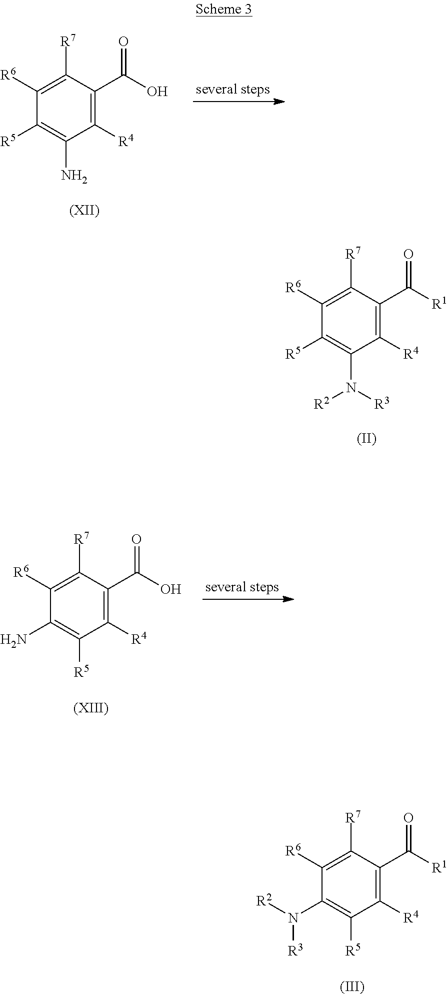

As shown in Scheme 4, a compound of formula IV, where each of R¹, R⁴, R⁵, R⁶, R⁷, R⁸, and R⁹ is as defined elsewhere herein, can be prepared from a compound formula IX by reaction with a ketone of formula XIV in a Friedländer-type synthesis (see Thummel, Synlett 1992, pg. 1; Cheng and Yan, Org. React. 28:37, 1982).

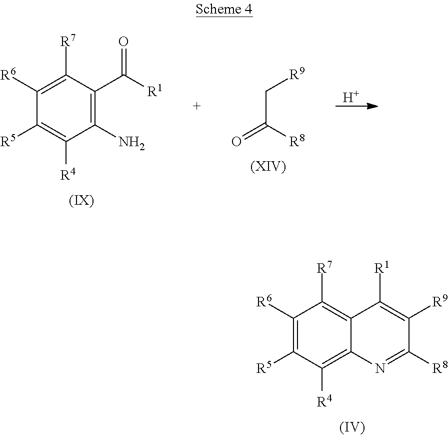

In some cases the chemistries outlined above may have to be modified, for instance, by the use of protective groups to prevent side reactions due to reactive groups, such as reactive groups attached as substituents. This may be achieved by means of conventional protecting groups as described in “Protective Groups in Organic Chemistry,” McOmie, Ed., Plenum Press, 1973 and in Greene and Wuts, “Protective Groups in Organic Synthesis,” John Wiley & Sons, 3^{rd} Edition, 1999.

### Formulation and Administration of a Composition of the Invention

A compound of formula I, formula II, formula III, or formula IV is preferably formulated into pharmaceutical compositions for administration to human subjects in a biologically compatible form suitable for administration in vivo. Accordingly, the present invention provides a pharmaceutical composition comprising a compound of formula I, formula II, formula III, or formula IV in admixture with a suitable diluent or carrier.

A compound of formula I, formula II, formula III, or formula IV may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The pharmaceutical compositions of the invention may be administered, for example, by oral, parenteral, buccal, sublingual, nasal, rectal, patch, pump, or transdermal administration and the compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A pharmaceutical composition of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a pharmaceutical composition of the invention may be incorporated with an excipient and used in the form of ingestible tablets, buccal tablets, troches, capsules, elixirs, suspensions, syrups, wafers, and the like.

A pharmaceutical composition of the invention may also be administered parenterally. Solutions of a composition of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Compositions suitable for buccal or sublingual administration include tablets, lozenges, and pastilles, where the active ingredient is formulated with a carrier, such as sugar, acacia, tragacanth, or gelatin and glycerin. Compositions for rectal administration are conveniently in the form of suppositories containing a conventional suppository base, such as cocoa butter.

The pharmaceutical compositions of the invention may be administered to an animal alone or in combination with pharmaceutically acceptable carriers, as noted above, the proportion of which is determined by the solubility and chemical nature of the compound, chosen route of administration, and standard pharmaceutical practice.

The dosage of the compositions of the invention, can vary depending on many factors, such as the pharmacodynamic properties of the active compound contained in the composition; the mode of administration; the age, health, and weight of the recipient; the nature and extent of the symptoms; the frequency of the treatment, and the type of concurrent treatment, if any; and the clearance rate of the active compound in the animal to be treated. One of skill in the art can determine the appropriate dosage based on the above factors. The pharmaceutical compositions of the invention may be administered initially in a suitable dosage that may be adjusted as required, depending on the clinical response. In general, satisfactory results may be obtained when the compositions of the invention are administered to a human at a daily dosage of active compound between 0.05 mg and 2000 mg (measured as the solid form). A desirable dose ranges between 0.05-30 mg/kg of active compound, more desirably between 0.5-20 mg/kg.

Administration of a composition of the invention may be as frequent as necessary to obtain the desired therapeutic effect. Some patients may respond rapidly to a higher or lower dose and may find much weaker maintenance doses adequate. Other patients, however, receive long-term treatments at the rate of 1 to 4 doses per day, in accordance with the physiological requirements of each patient.

The following non-limiting examples are provided to further describe various aspects and embodiments of the present invention.

### EXAMPLES

### Bacterial Strains, Plasmids, and Media

P. aeruginosa strains include wild-type (PA14, see Rahme et al., Science 268:1899-1902, 1995); a PA14 mvfR mutant (Cao et al., Proc. Natl. Acad. Sci. USA 98:14613-14618, 2001); a pqsE deletion mutant, generated via pEX18Ap allelic replacement by using sucrose selection (Hoang et al., Gene 212:77-86, 1998), resulting in a 570-bp nonpolar deletion covering 65% of the sequence (Déziel et al. PNAS); and the pqsA (U479) TnphoA mutant obtained from the PA14 Transposon Insertion Mutant Database. Bacteria were grown in LB broth or on 1.5% Bacto-agar (Difco) LB plates. Freshly plated cells served as inoculum.

### LC/MS Analysis

Analyses were performed by using a Micromass Quattro II triple quadrupole mass spectrometer (Micromass Canada, Pointe-Claire, Canada) in positive electrospray ionization mode, interfaced to an HP 1100 HPLC equipped with a 4.5×150-mm reverse-phase C₈ column. Culture supernatants were twice extracted with ethyl acetate, the solvent was evaporated, and the residue was dissolved in a water/acetonitrile mixture containing the internal standard. Alternatively, culture samples were directly diluted with a methanolic solution of the internal standard, as previously reported (Lépine et al., Biochim. Biophys. Acta 1622:36-40, 2003).

### Burn/Infection Model

Burn wound infection in mice can be established by subcutaneous or topical administration of the bacteria to the sites of the burn. In order to demonstrate the anti-microbial activity of the pharmaceutical compositions of the present invention, the ability of such compositions to prevent burn wound infection was studied. Six-week old CD-1 male mice were used.

To induce infection, the mice were placed under general anesthesia via intraperitonial injection of 40 mg/kg Pentobarbital, the ventral surface was shaved, and a central skin fold elevated was simultaneously compressed from both sides for 5 seconds using two 2×2 cm metal blocks preheated to 95-100° C., to produce a non-lethal 5-8% full thickness burn with negligible focal necrosis of the underlying muscle or damage to underlying visceral organs. IP saline was administered for fluid resuscitation for ≧5% total body surface burns: avoiding vital structures, saline was injected into the left lower quadrant of the abdominal area of the anesthetized animal post-burn. This burn procedure was followed by mid-eschar inoculation of 100 μL of bacterial suspension containing ˜5×10⁵ CFU. The animals were returned to their cages and provided mouse chow and water ad libitum. Animals typically recover from the anesthesia within 20 minutes post-administration. Criteria used to evaluate anesthesia recovery are state of alertness, mobility, and reaction to surrounding stimuli. Mice were monitored twice a day for symptoms and deaths, with increased monitoring every 6-8 hours during the period of greatest potential for infection and clinical disease.

2-aminoacetophenone (2AA), which is naturally produced by P. aeruginosa, has immunomodulatory activity in mice, and can be used to “prime” host defenses to restrict at least P. aeruginosa infections. Recent data also show that 2AA may also have anti-inflammatory properties, as it restricts over-activation of inflammation in response to burn, and burn and P. aeruginosa infection.

Bacterial-host interactions are mediated by large sets of QS-controlled bacterial virulence factors. Intrinsic bacterial compounds and/or their metabolites can enhance the immune response against P. aeruginosa in infected patients. The results presented below demonstrate that 2AA is one such molecule. LC/MS analysis of P. aeruginosa supernatant shows that 2AA production peaks at the late stationary phase of bacterial growth. Indeed, 2AA production is controlled by the QS transcriptional activator MvfR. We have demonstrated, as discussed below, that 2AA is an immunomodulatory compound based on the fact that: 1) 2AA pre-treatment of mice greatly increases their survival to P. aeruginosa pathogenesis, using burn and infection, and lung infection, models; 2) this protection effect is specific, as 2AA analogs and a metabolite provide significantly less protection; 3) pilot whole-genome expression experiments show 2AA modulates the innate immune system in both naive, and burn and P. aeruginosa-infected, mice; 4) 2AA treatment initially induces inflammatory response functions priming the animals, as this induction is protective to subsequent P. aeruginosa infection; and 5) pre-exposure to 2AA restricts over-activation of inflammation, which can further increase host damage and mortality, in response to burn, and burn and infection and thus may have anti-inflammatory activity in these animals.

### Example 1

### 2AA Production in P. Aeruginosa

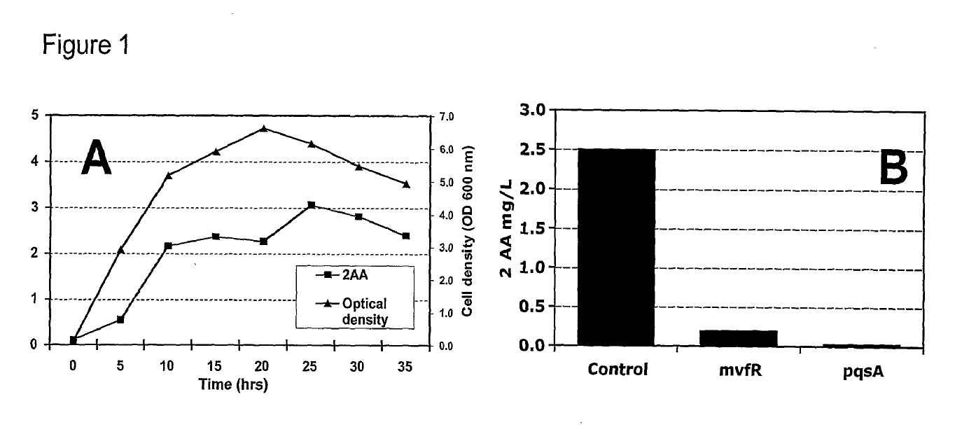

To identify QS compounds with immunomodulatory and/or anti-infective activity, we investigated if 2AA plays a role in P. aeruginosa pathogenesis. 2AA is secreted in the spent medium of P. aeruginosa cultures. LC-MS analysis of bacterial supernatants at different growth time points shows that this compound maximally accumulates during the stationary phase of bacterial growth (FIG. 1A), indicating it may be QS regulated. The P. aeruginosa transcriptional factor MvfR controls the production of QS-dependent molecules, and FIG. 1B shows that 2AA production also requires MvfR. Furthermore, pqsA mutant cells, which fail to make hydroxyalkylquinolones (HAQs), also do not produce 2AA, suggesting it is also a product of the HAQ biosynthetic pathway. To this end, we supplemented PA14 cultures with deuterium-labeled HHQ and anthranilic acid (AA), the precursor of all HAQs, and found labeled 2AA is produced only from labeled AA.

### Example 2

### Mass Spectrometry Experiments of 2′-Aminoacetophenone Provided to PA and PA Mutants

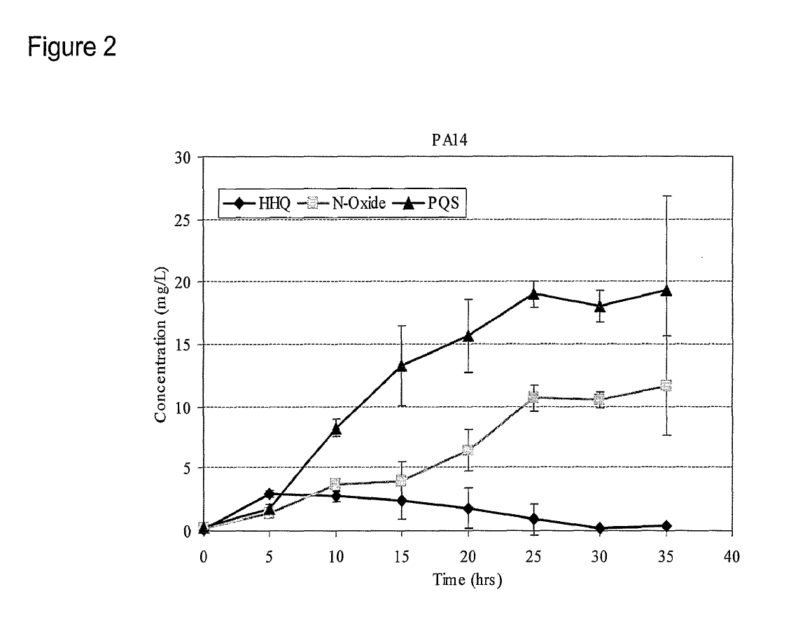

Deuterium-labeled 2-aminobenzoic acid (AA-d₄, available from CDN Isotopes, Pointe-Claire, Canada) or deuterium-labeled 4-hydroxy-2-heptylquinoline (HHQ-d₄, prepared by the procedure of Lépine et al., Biochim, Biophys, Acta 1622:36 40, 2003) was fed to wild type (WT), MvfR mutant, pqsE mutant, or pqsA PA mutant cultures. FIG. 2 shows the levels of HAQs produced by wild type. The presence of labeled-2′-aminoacetophenone was then assessed by LC/MS analysis (M+H⁺=136). In the WT cultures it was observed that labeled 2′-aminoacetophenone was produced only from AA-d₄ and not from HHQ-d₄, indicating that 2′-aminoacetophenone originates from 2-aminobenzoic acid, which is largely generated by the action of the MvfR-controlled PhnAB anthranilate synthase. In the mutant cultures, it was found that the mvfR, phnA, and pqsA mutants were unable to produce 2′-aminoacetophenone while the pqsE mutant is still able to produce this compound.

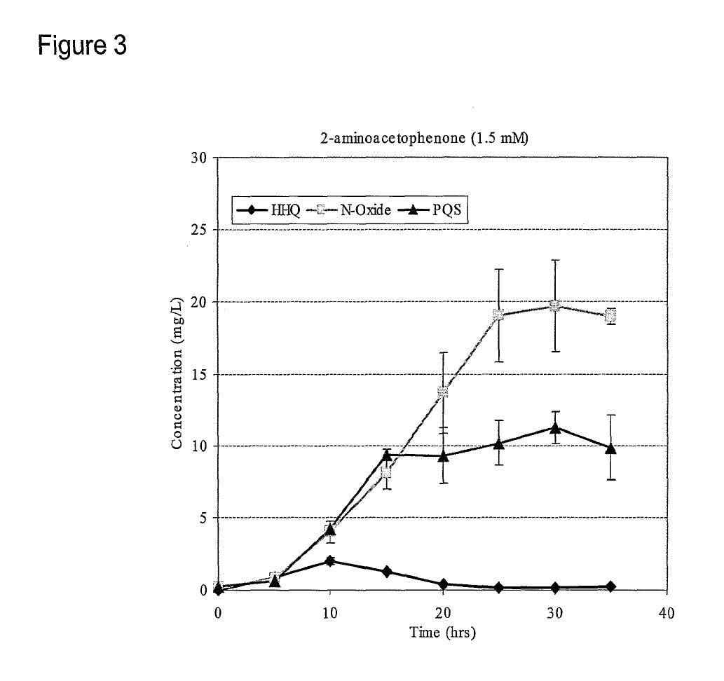

In a separate experiment, decreased the production of 4-hydroxy-2-heptylquinoline (HHQ, M+H⁺=244) and 3,4-dihydroxy-2-heptylquinoline (PQS, M+H⁺=260) but enhanced the production of 4-hydroxy-2-heptylquinoline N-oxide (HQNO, M+H⁺=260) (see FIG. 3). These findings suggest that 2′-aminoacetophenone may function as an anti-infective agent and that it is produced by PA to regulate its own pathogenicity by switching-off virulence networks and/or activating the production of bacterial homeostatic compounds (such as, e.g., N-oxides) that also have anti-microbial activity.

### Example 3

### P. Aeruginosa Growth Inhibition Experiments

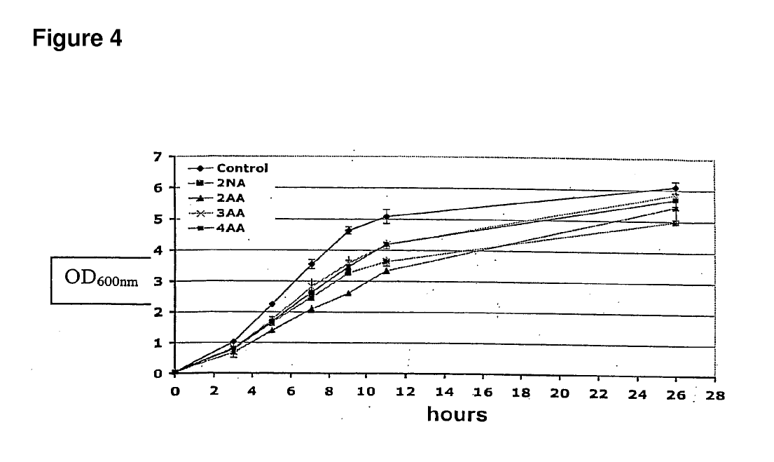

P. aeruginosa was grown in the presence of 2 mM of 2′-aminoacetophenone (2AA), 3′-aminoacetophenone (3AA), 4′-aminoacetophenone (4AA), or 2′-nitroacetophenone (2NA), or in the absence of any of these compounds (control). Bacterial growth was followed over 26 hours by spectrophotometric analysis at 600 nm. As shown in FIG. 4, none of these compounds appear to have anti-bacterial activity as bacterial growth was only slightly retarded, but no bacteria were killed.

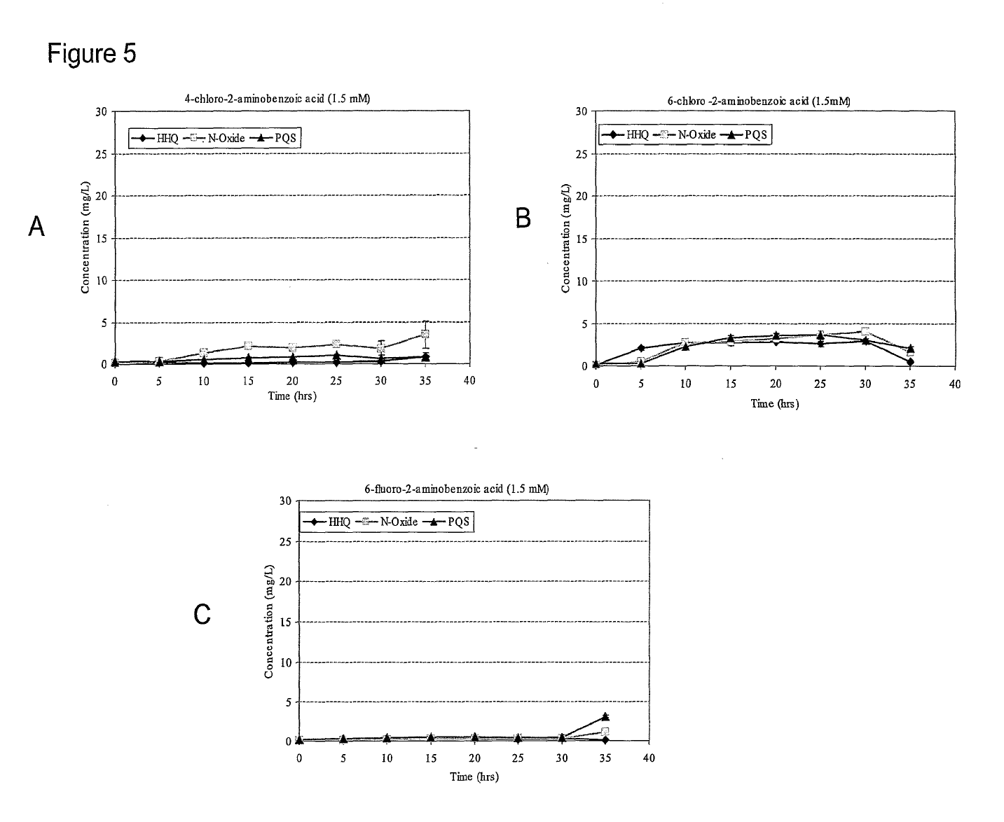

P. aeruginosa was also grown in the presence of 1.5 mM of 6-fluoro-2-aminobenzoic acid, 5-fluoro-2-aminobenzoic acid, 4-fluoro-2-aminobenzoic acid, 6-chloro-2-aminobenzoic acid, and 4-chloro-2-aminobenzoic acid, or in the absence of any of these compounds (FIG. 5 control A and control B). These compounds were found to be potent inhibitors of 4-hydroxy-2-alkylquinoline (HAQ) production. When the treated P. aeruginosa bacterial cultures were analyzed for HAQs by mass spectral analysis, the production of each of the principal members of the three main families of HAQs (4-hydroxy-2-heptylquinoline (HHQ), 4-hydroxy-2-heptylquinoline N-oxide (HQNO), and 3,4-dihydroxy-2-heptylquinoline (PQS)) was dramatically decreased (see FIGS. 5A, 5B, and 5C for the results of cultures treated with 4-chloro-2-aminobenzoic acid, 6-chloro-2-aminobenzoic acid, and 6-fluoro-2-aminobenzoic acid, respectively).

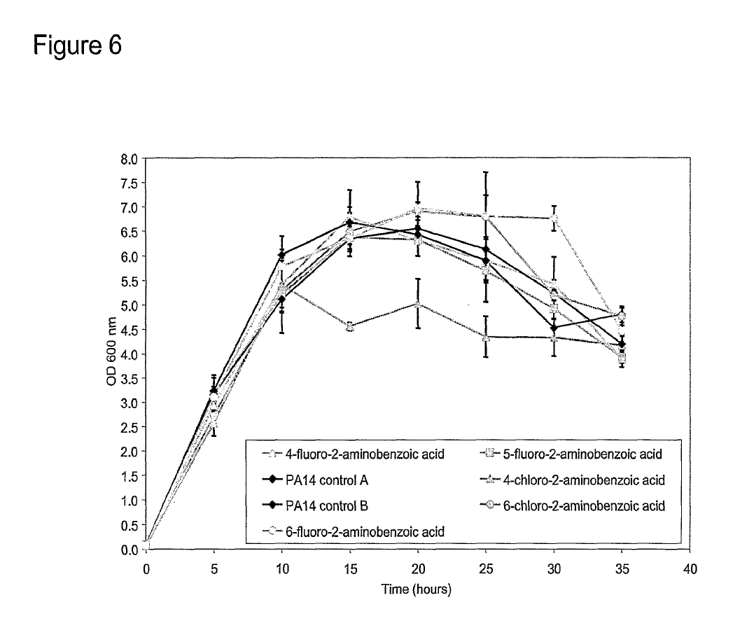

Bacterial growth was also followed over 35 hours by spectrophotometric analysis at 600 nm. As shown in FIG. 6, none of these compounds appear to have anti-bacteriocidal activity against P. aeruginosa as bacterial growth was only slightly retarded in the experiment using 4-chloro-2-aminobenzoic acid.

### Example 4

### 2AA Protects Burn Injured Mice Against P. Aeruginosa Infection

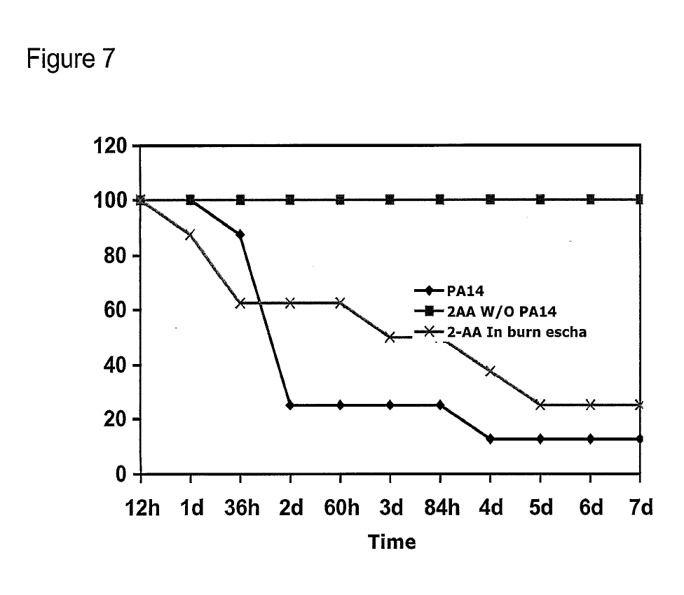

The mouse full-thickness skin thermal injury infection model was used to test if 2AA restricts P, aeruginosa virulence. This model is predictable and reproducible; clinically relevant; and can lead to MODS and mortality. Briefly, a ˜15% total burn surface area (TBSA) thermal scalding injury is produced on the abdominal skin, leaving the underlying muscle intact, and the inoculum is delivered into the scald eschar. Using this model, 2AA was injected in the scald eschar 1 hr post burn and infection with PA14 (burn and infection: “BI”). FIG. 7 shows that mice that received 2AA succumb to PA14 lethality significantly later than the untreated BI mice, indicating 2AA limits P. aeruginosa virulence.

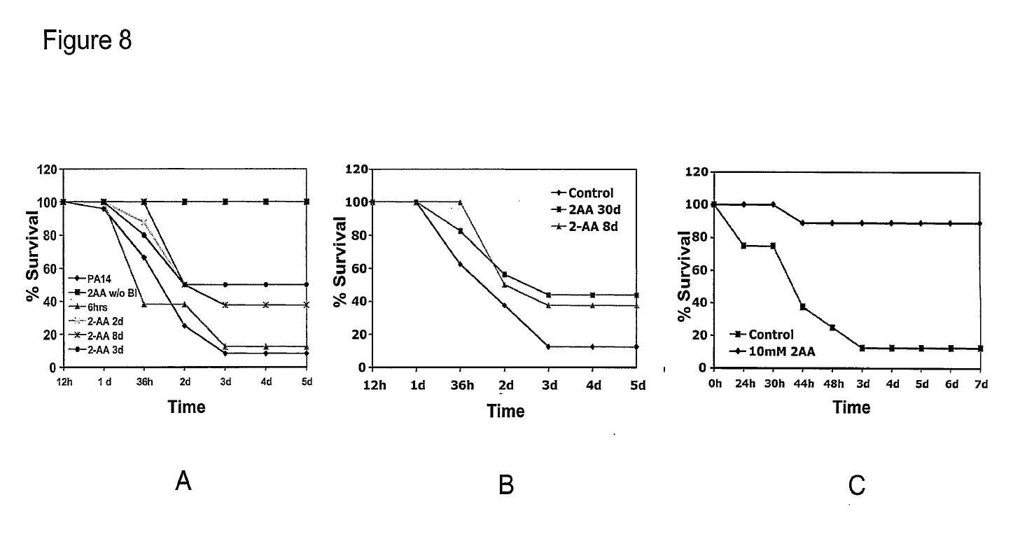

In another set of experiments we first pre-exposed mice to 2AA prior to BI treatment. FIG. 8A shows that pretreatment with a single intraperitonealy (IP) injection of 500 μl of 20 mM 2AA at 6 hours pre-BI provides no protection, and animals die at the same rate as the uninjected BI controls. In contrast, increased mice survival is found if the 2AA injection occurs 2 days pre-BI: these mice exhibit 60% mortality versus the ˜90% mortality of the controls (FIG. 8A). Interestingly, survival is further increased to 50% when 2AA injection occurs 3 or 8 days pre-BI (FIG. 8A); and this protection effect is long lasting, as mice injected with 2AA up to 4 weeks pre-BI still exhibit 50% mortality (FIG. 8B). Most remarkably, mice injected intravenously (IV), versus IP, with 100 μl of 10 mM 2AA 4 days pre-BI, show ˜90% survival to PA14 infection (FIG. 8C).

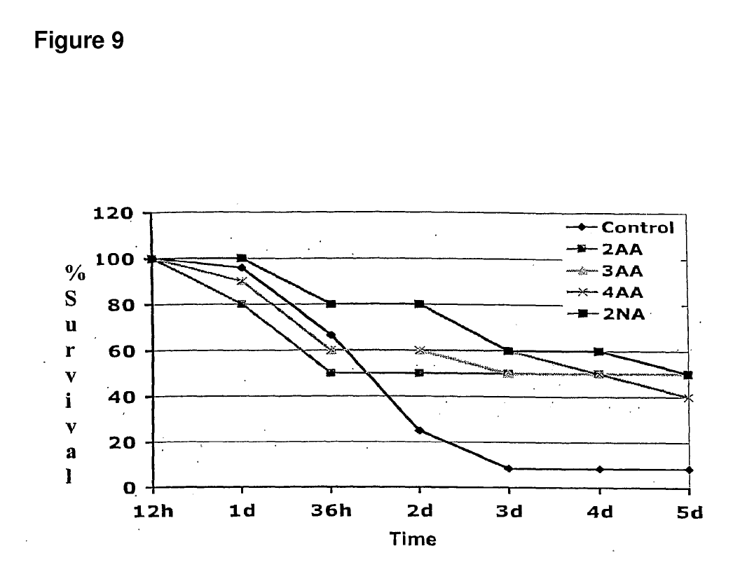

In another experiment to determine the prophylactic effect of 2′-aminoacetophenone using the mouse burn/infection model, the results of which are shown in FIG. 9, mice were treated intravenously through the tail vein with 100 μl of a 10 mM solution of 2′-aminoacetophenone (2AA), 3′-aminoacetophenone (3AA), 4′-aminoacetophenone (4AA), or 2′-nitroacetophenone (2NA) four days prior to burn/infection injury (n=8-10 for each compound) show an average of 50% survival after 5 days compared to 10% survival in control animals treated with only 0.9% saline.

### Example 5

### Structural Analogs Demonstrate the Specificity of the 2AA Protection Effect

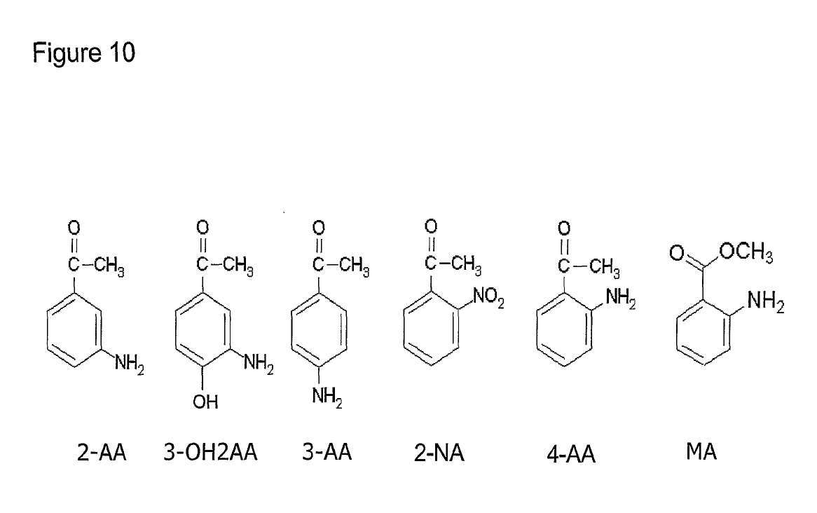

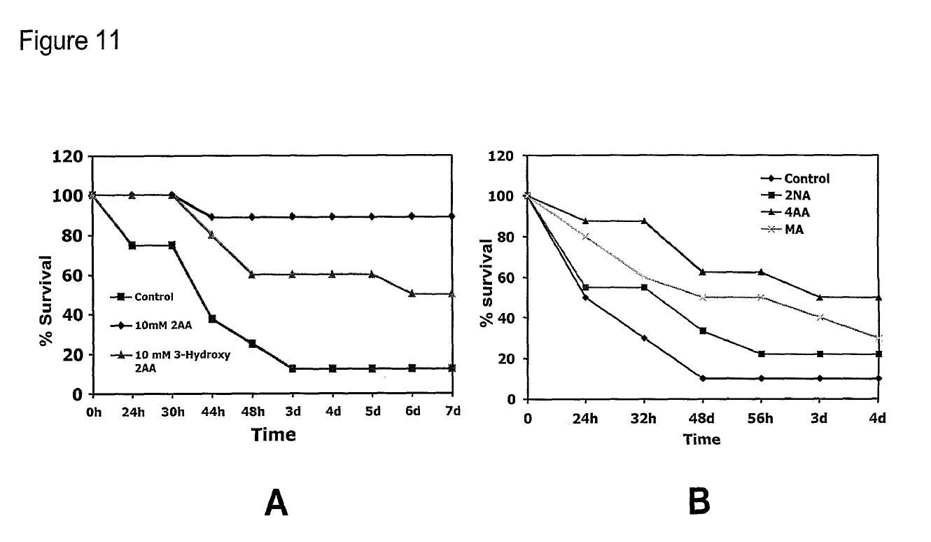

We next tested the protection efficacy of four 2AA structural analogs (FIG. 10): 3-amino acetophenone (3AA) and 4-amino acetophenone (4AA) have an amino group at the 3 or 4 position of the ring; 2-nitro acetophenone (2NA), has a nitro group substitution of the position 2 amino group; and methyl anthranilate (MA) contains a methyl group in place of hydrogen in the AA carboxyl group, versus the 2AA acetyl group. Ten millimolar (10 RIM) solutions of these compounds were IV injected into mice 4 days pre-BI, and FIGS. 11A and 11B show that the protection afforded by 2AA is significantly greater than those of its analogs. Because 2AA is metabolized in vivo into 2-amino-3 hydroxy acetophenone (FIG. 10), we chemically synthesized this compound and assayed it in mice. FIG. 11A shows that it also has decreased protective efficacy versus 2AA.

### Example 6

### Protection of Mice Pre-Treated with 2AA in Lung Infection Model

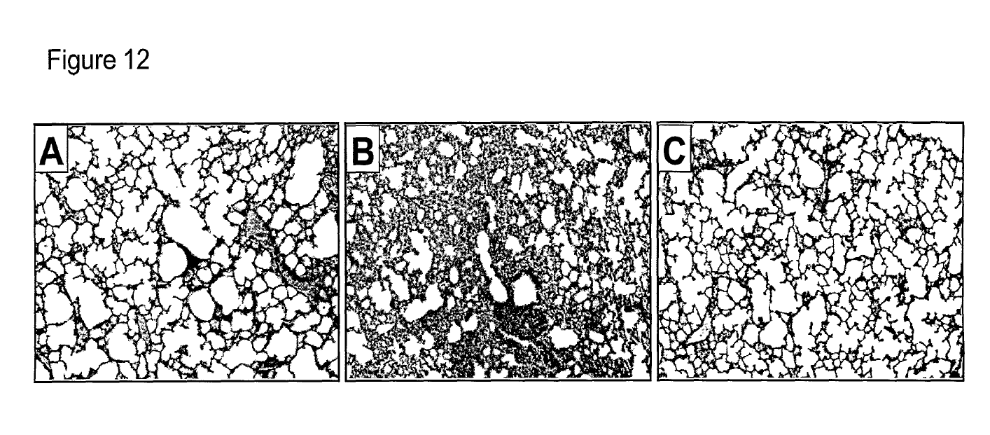

We next assessed the morphological dynamics of PA14 infection in the presence of 2AA in a neonatal mouse lung infection model (Tang et al., Infection & Immunity 64:37-43, 1996), which is relevant to CF. A minimum inoculum of 1.5×10⁵ PA14 cells/animal is 100% lethal in this model. Mice were treated with 2AA 4 days pre-infection, sacrificed at 24, 48, and 72 hr post-infection, and lung histopathology was assessed and compared to that of controls. PA14 infection alone produces rapidly developing inflammation that by 24 hr presents with red hepatization and development of lobular pneumonia. By 48 hours, the lung pathology includes most of the parenchyma with extensive formation of bacteria-filled necrotic foci (FIG. 12B). These observations are also consistent with our previous mortality studies using this lung model that indicate 100% mortality by 60 hr. In contrast, mice pretreated with 2AA exhibit greatly delayed rates of development of inflammation (compare FIG. 12A with FIG. 12C), in response to PA14 infection. Note that the inability of PA14 cells in the presence of 2AA to progress beyond interstitial involvement indicates that PA14 does not impair the host immune response, which is capable of clearing infections at early stages.

### Example 7

### 2AA Inhibits the Production of HHQ and PQS

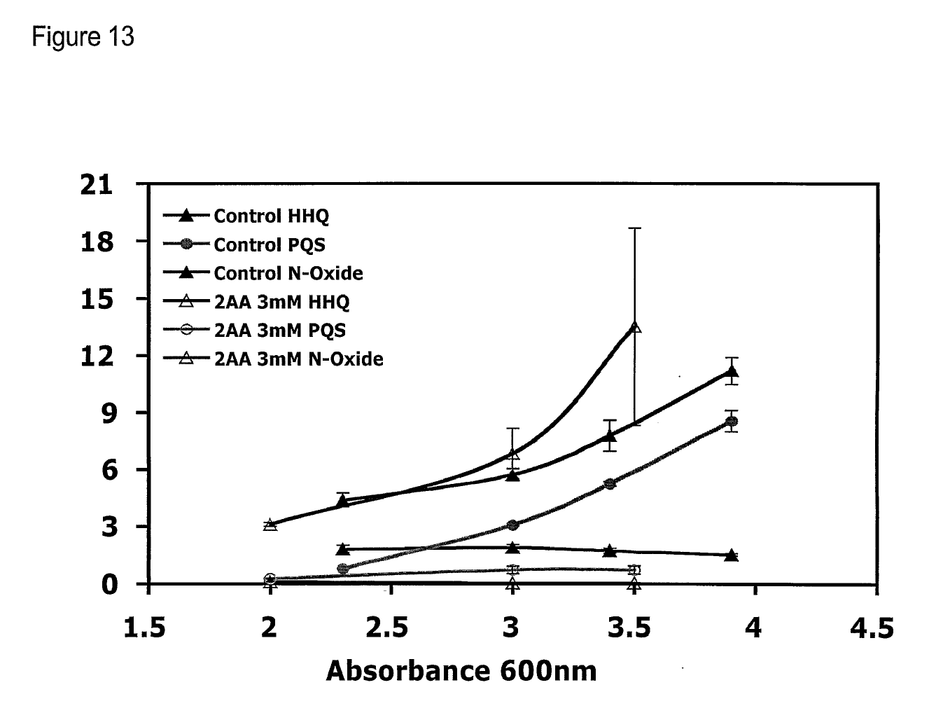

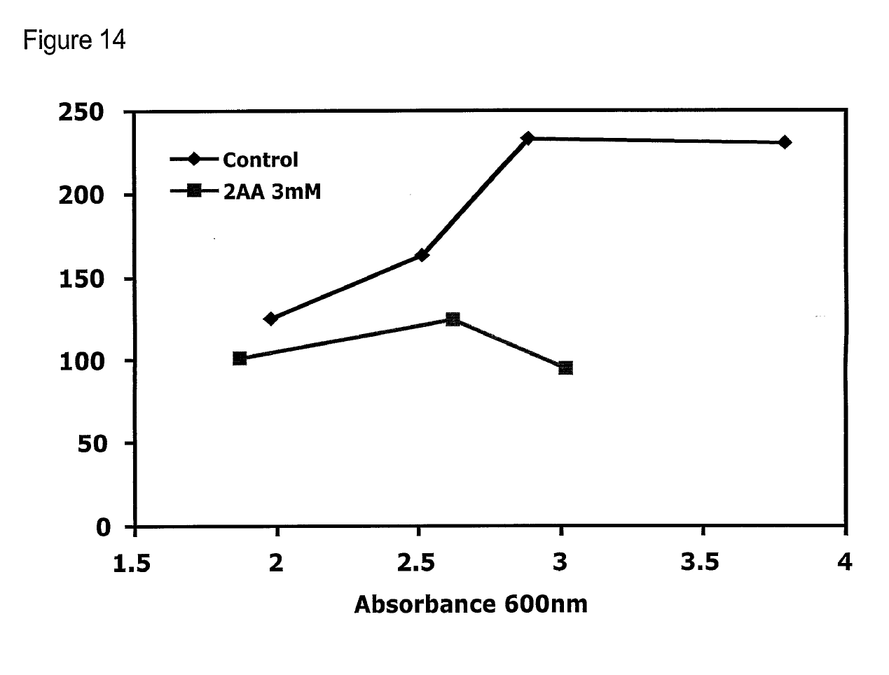

We have found that 2AA reduces mouse mortality when is injected in the burn eschar of burned and P. aeruginosa infected animals (FIG. 7). To assess the effect of this compound on bacteria, we analyzed the production of HAQs in the presence of 2AA using LC/MS. The bacteria were propagated in the presence of 3 mM 2AA, culture media was collected at different time points, and the HHQ, PQS and HQNO production was measured in the supernatant. FIG. 13 shows that in presence of 3 mM 2AA no HHQ was produced and only a limited quantity of PQS was produced. On the contrary, however, there was higher accumulation of HQNO compared to the bacteria grown without 2AA. Since 2AA addition to the P. aeruginosa culture inhibits the production of HHQ and PQS, we assessed the transcription from the pqs operon using a pqsA-LacZ fusion. As shown in FIG. 14, in presence of 2AA there is a significant reduction in LacZ expression.

### Example 8

### 2AA Competes with the MvfR Ligands HHQ and PQS

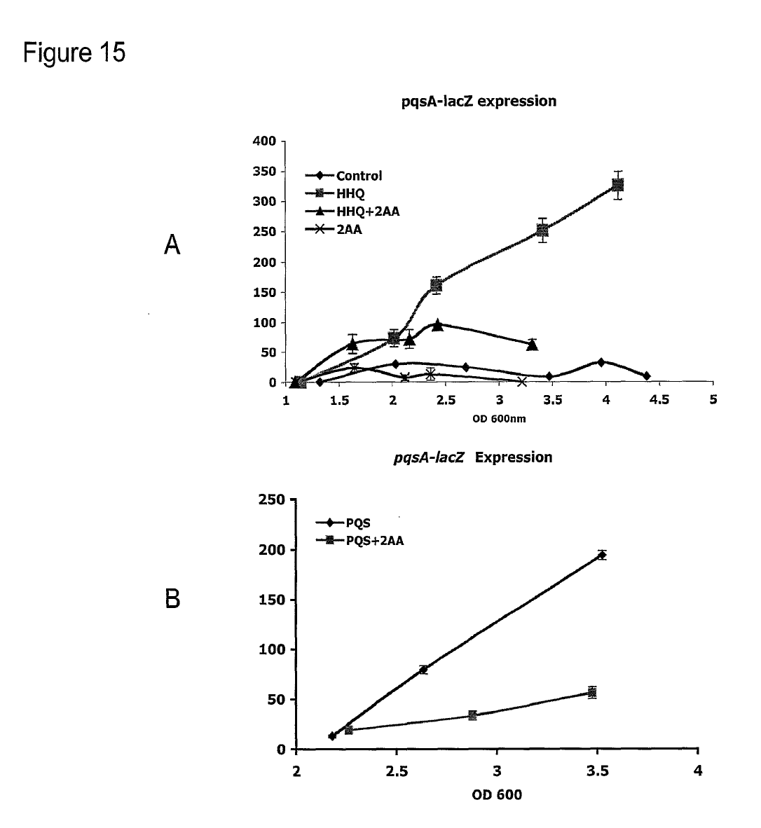

To further, understand the mechanism of inhibition of the pqs operon by 2AA, we carried out competition assays of PQS and HHQ with 2AA. The expression of pqsA-LacZ in pqsApqsH double mutants was assessed. Bacteria were grown to an OD₆₀₀ of 1.0 and HHQ and PQS were added either alone or with 2AA. LacZ expression was assayed at different stages of the growth, using standard protocols. As shown in FIGS. 13, 15A, and 15B, addition of 3 mM 2AA in cultures containing 1 mg/L HHQ or 10 μl PQS blocks pqsA expression. However, 2AA addition in cultures containing exogenously added PQS inhibits pqsA expression at later time points. These results indicate that 2AA inhibits pqsA expression by competing with the MvfR ligands, HHQ and PQS, which are required for its activation.

We also carried out whole genome expression studies of P. aeruginosa cells in the presence of 3 mM 2AA. Cells were grown in presence of 3 in M 2AA and harvested at an OD₆₀₀ of 3.0. Culture without 2AA was used as control. Total RNA was extracted and converted to cDNA. The labeled cDNA was used to hybridize to a whole genome P. aeruginosa Affymetrix chip. Genes whose expression was altered more than 2 fold in presence of 2AA were selected. We detected approximately 1250 genes whose expression was altered following 2AA addition (1056 downregulated and 199 upregulated). The expression of many of these genes is controlled by MvfR, confirming that 2AA inhibits the MvfR pathway, likely by competing with the binding of the MvfR ligands HHQ and PQS. Genes listed in Table 1 are the ones that are also affected by loss of MvfR function.

TABLE 1
Genes downregulated by 2AA

| | FOLD | |
|---|---|---|
| GENE | CHANGE | DESCRIPTION |
| PA0122 | −3.4 | conserved hypothetical protein |
| PA0355 | −2.7 | protease pfpI |
| PA0567 | −3.0 | conserved hypothetical protein |
| PA0905 | −2.7 | carbon storage regulator csrA |
| PA0996 | −3.0 | probable coenzyme A ligase pqsA |
| PA0997 | −4.6 | hypothetical protein pqsB |
| PA0998 | −5.4 | hypothetical protein pqsC |
| PA0999 | −5.2 | 3-oxoacyl-[acyl-carrier-protein] synthase |
| | | III pqsD |
| PA1000 | −4.3 | hypothetical protein pqsE |
| PA1001 | −6.2 | anthranilate synthase component I, phnA |
| PA1002 | −4.9 | anthranilate synthase component II, phnB |
| PA1216 | −2.7 | hypothetical protein |
| PA1456 | −2.3 | two-component response regulator CheY |
| PA1457 | −2.1 | chemotaxis protein CheZ |
| PA1852 | −2.3 | hypothetical protein |
| PA1901 | −3.4 | phenazine biosynthesis protein PhzC |
| PA1902 | −3.4 | phenazine biosynthesis protein PhzD |
| PA1903 | −3.1 | phenazine biosynthesis protein PhzE |
| PA1904 | −3.6 | probable phenazine biosynthesis protein |
| PA1905 | −3.4 | probable pyridoxamine 5′-phosphate oxidase |
| PA1914 | −26.3 | conserved hypothetical protein |
| PA2031 | −2.0 | hypothetical protein |
| PA2067 | −2.5 | probable hydrolase |
| PA2069 | −2.5 | probable carbamoyl transferase |
| PA2134 | −2.5 | hypothetical protein |
| PA2172 | −2.4 | hypothetical protein |
| PA2173 | −2.3 | hypothetical protein |
| PA2194 | −3.5 | hydrogen cyanide synthase HcnB |
| PA2195 | −2.9 | hydrogen cyanide synthase HcnC |
| PA2204 | −3.1 | probable component of ABC transporter |
| PA2274 | −3.7 | hypothetical protein |
| PA2300 | −3.2 | chitinase |
| PA2327 | −4.9 | probable permease of ABC transporter |
| PA2328 | −4.5 | hypothetical protein |
| PA2329 | −9.8 | probable ATP-binding component of ABC |
| | | transporter |
| PA2330 | −11.7 | hypothetical protein |
| PA2331 | −10.9 | hypothetical protein |
| PA2486 | −2.3 | hypothetical protein |
| PA2570 | −4.5 | PA-I galactophilic lectin |
| PA2754 | −2.0 | conserved hypothetical protein |
| PA3031 | −3.1 | hypothetical protein |
| PA3096 | −2.8 | general secretion pathway protein L |
| PA3098 | −2.2 | general secretion pathway protein J |
| PA3099 | −2.2 | general secretion pathway protein I |
| PA3100 | −2.1 | general secretion pathway protein H |
| PA3101 | −3.4 | general secretion pathway protein G |
| PA3105 | −2.2 | general secretion pathway protein D |
| PA3126 | −2.5 | heat-shock protein IbpA |
| PA3186 | −2.2 | outer membrane porin OprB precursor |
| PA3187 | −2.6 | probable ATP-binding component of ABC |
| | | transporter |
| PA3189 | −2.0 | probable permease of ABC sugar transporter |
| PA3195 | −3.2 | glyceraldehyde 3-phosphate dehydrogenase |
| PA3370 | −2.2 | hypothetical protein |
| PA3371 | −2.1 | hypothetical protein |
| PA3569 | −2.6 | 3-hydroxyisobutyrate dehydrogenase mmsB |
| PA3570 | −2.9 | methylmalonate-semialdehyde dehydrogenase |
| PA3691 | −7.0 | hypothetical protein |
| PA3692 | −4.7 | probable outer membrane protein |
| PA3812 | −5.9 | probable iron-binding protein IscA |
| PA3813 | −6.7 | probable iron-binding protein IscU |
| PA4205 | −6.1 | hypothetical protein MexG |
| PA4206 | −5.9 | probable RND efflux membrane fusion protein |
| | | precursor mexH |
| PA4207 | −7.5 | probable RND efflux transporter mexI |
| PA4208 | −6.8 | probable outer membrane efflux protein |
| | | precursor, OpmD |
| PA4209 | −2.4 | probable O-methyltransferase phzM |
| PA4210 | −3.3 | probable phenazine biosynthesis protein phzA1 |
| PA4211 | −2.9 | probable phenazine biosynthesis protein phzB1 |
| PA4217 | −5.4 | probable FAD-dependent monooxygenase phzS1 |
| PA4218 | −5.3 | probable transporter |
| PA4739 | −3.7 | conserved hypothetical protein |
| PA4876 | −2.6 | osmotically inducible lipoprotein OsmE |
| PA4917 | −2.1 | hypothetical protein |

### Example 9

### Anti-Infective and Immunomodulatory Compounds Directed to MvfR

The virulence-related transcriptional regulator MvfR is a potential target for anti-infection therapeutics, as it plays a central role in the control of quorum sensing (QS)-controlled genes in Pseudomonas aeruginosa; it directs the synthesis of signal molecules that modulate the expression of a large array of virulence-related QS-controlled genes; and its activation is mediated via its binding to specific ligands essential for its function. Thus, we sought to restrict the activity of the MvfR/HAQ regulatory pathway. To this end, we have 1) identified the MvfR regulatory factors and their mode of action by confirming the identity of MvfR ligands, their binding to MvfR, and their mechanism of action; and 2) designed molecules that perturb MvfR activity by inhibiting the synthesis of its co-ligands.

Identification of the Co-Ligands that Bind to MvfR, and Demonstration that they Modulate MvfR Regulatory Activity

By analogy with other members of the LTTR family of transcriptional regulators, the MvfR C-terminal regulatory domain should bind a specific ligand that mediates its activation. Indeed, the C-terminal region between positions 92-298 of MvfR encodes a predicted ligand-binding domain (LBD). LTTR ligands typically are related to the primary function of their respective regulator, and are often a substrate or product of the metabolic pathway controlled by this regulator (Schell, Ann. Rev. Microbiol. 47:597-626, 1993). Since a principle MvfR function is to control HAQ biosynthesis, we hypothesized the MvfR ligand is an HAQ, such as PQS, or an HAQ derivative. Moreover, the fact that LBD mutations affect MvfR function and PQS induces MvfR activity in a pqsA mutant background indicates that PQS and the MvfR LBD play important roles in regulating MvfR activity. The identification of MvfR ligands and their mechanism of action allowed us to test MvfR-based strategies for limiting P. aeruginosa pathogenesis.

### Both HHQ and PQS are Inducers of the P. Aeruginosa Transcription Factor MvfR

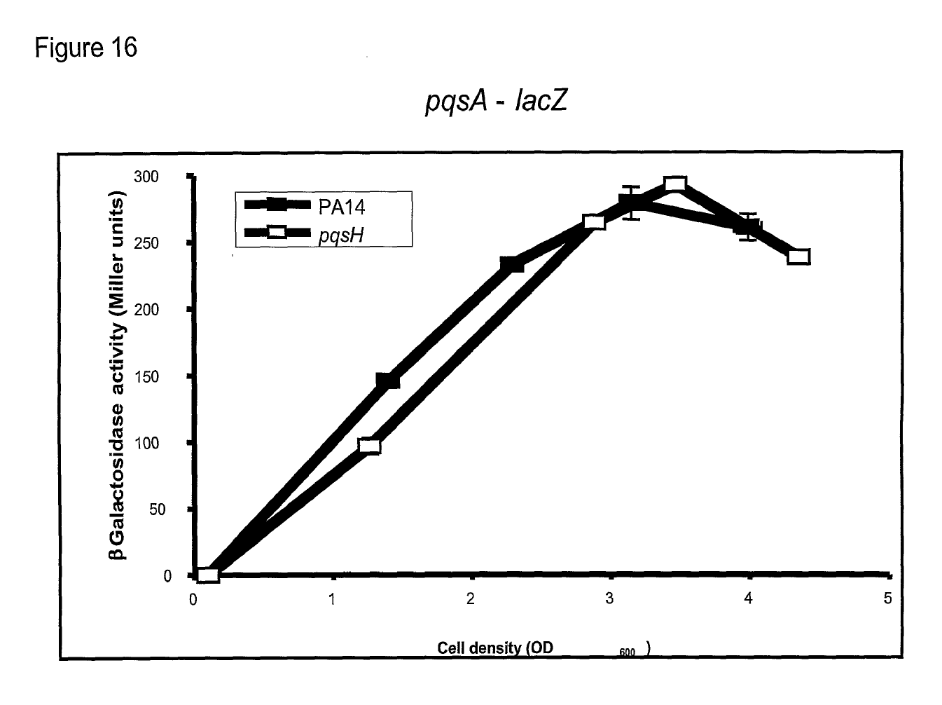

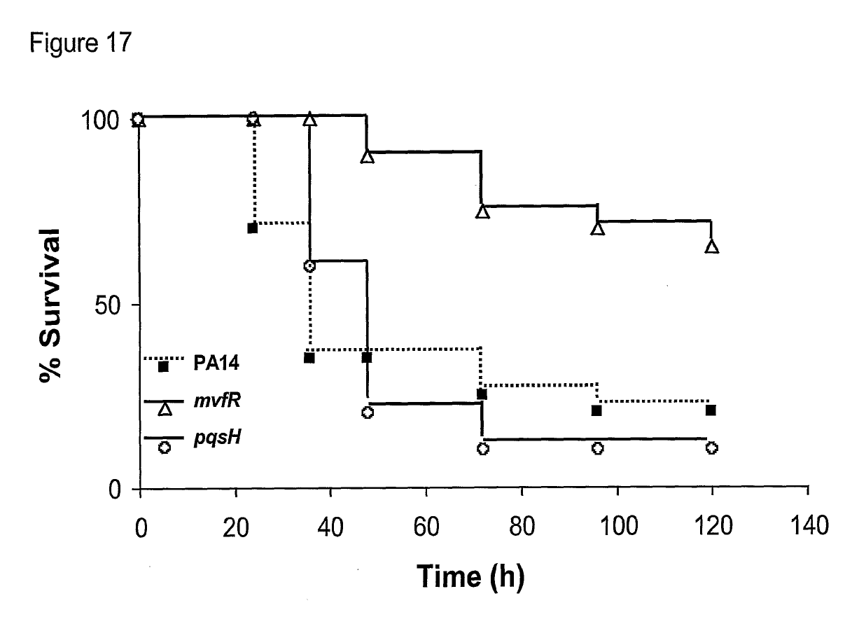

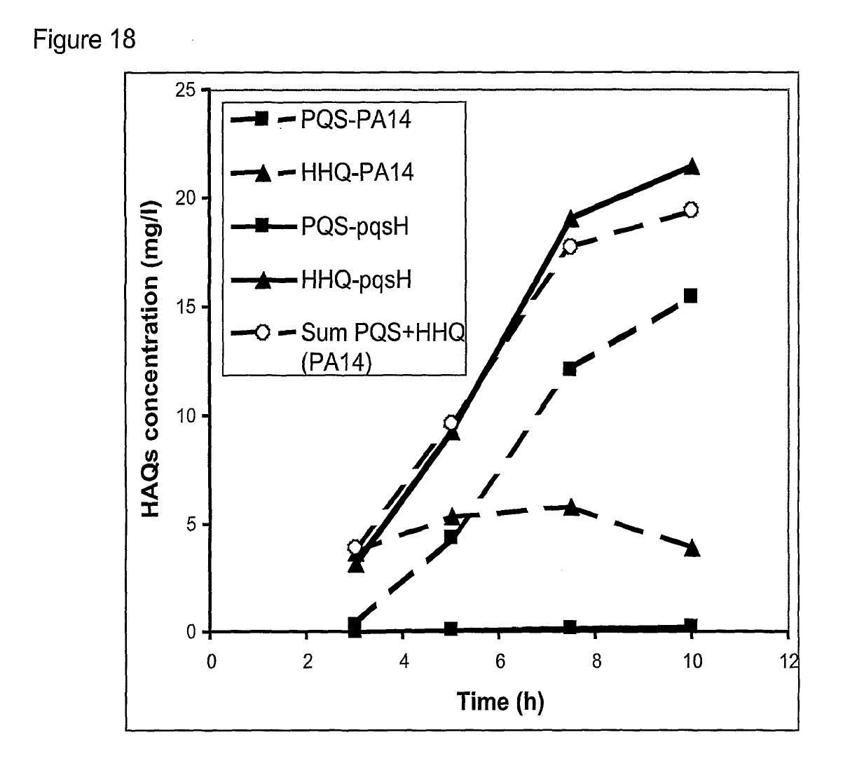

PQS binds to MvfR and activates pqsA transcription in pqsA mutant cells that lack all HAQ production. Furthermore, PQS enhances the in vitro binding of MvfR to the pqsA-E promoter, indicating that PQS is a MvfR co-inducer. Conversely, our studies show that PQS is not required for MvfR to activate pqsA-E, which indicates that another molecule also functions in vivo as an mvfR co-inducer. Our data show: 1) pqsA-lacZ expression is fully activated in psqH cells (FIG. 16), which completely lack PQS (PqsH catalyzes the final step in PQS biosynthesis; Déziel et al., P.N.A.S. USA 101:1339-1344, 2004); 2) PqsH cells exhibit normal virulence, in contrast to mvfR cells (FIG. 17); and 3) PqsH cells produce other HAQs that activate MvfR (FIG. 18).

In addition, liquid chromatography coupled to mass spectrometry (LC/MS) data show psqH mutant cells completely lack PQS and accumulate increased levels of HHQ, a major HAQ product of the pqsA-D biosynthetic pathway.

To determine whether HHQ activates MvfR expression, we generated a PA14pqsH/pqsA double mutant that lacks all HAQ production, and assessed pqsA-lacZ activity in the absence and presence of HHQ and PQS.

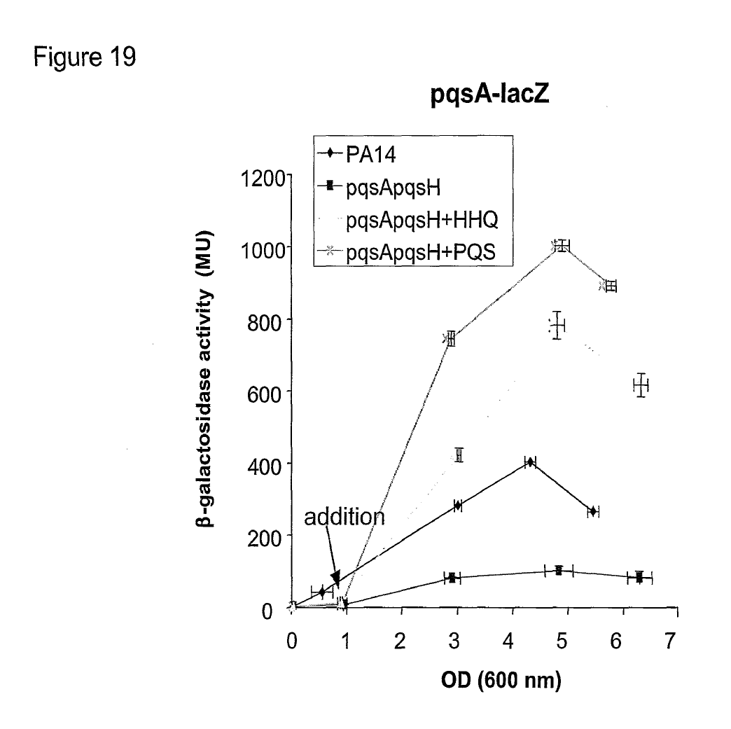

FIG. 19 shows that no significant pqsA-lacZ activity occurs in the absence of PQS and HHQ. Thus, these compounds induce MvfR-dependent pqsA-lacZ activity. Furthermore, MvfR activation of pqsA-E does not require PQS, as HHQ can also act as an MvfR co-inducer (FIG. 16).

### Both HHQ and PQS Act as MvfR Ligands

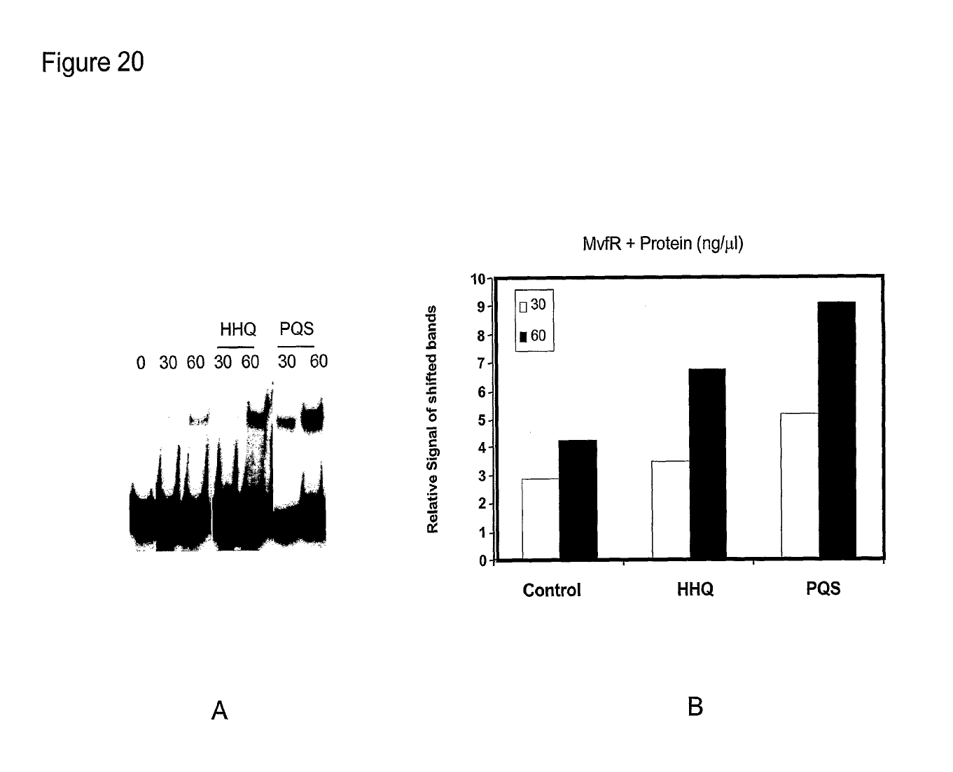

We previously demonstrated that MvfR binds to the phnA promoter in the absence of ligand (Cao et al., P.N.A.S. USA 98:14613-14618, 2001). To determine if HHQ potentiates MvfR DNA-binding, a 174-bp radiolabeled pqsA promoter region DNA fragment containing the potential LysR-box sequence was mixed with E. coli cell lysate containing MvfR. FIGS. 20A and 20B show: 1) MvfR binds this DNA fragment in the absence of HHQ or PQS; and 2) both these compounds potentiate MvfR-pqsA promoter binding, with weaker activity seen with HHQ versus PQS. These results demonstrate a novel role for HHQ in QS regulation and cell-cell signaling.

### HHQ Functions as an MvfR Co-Inducer to Activate Pqs-Operon Expression in E. Coli

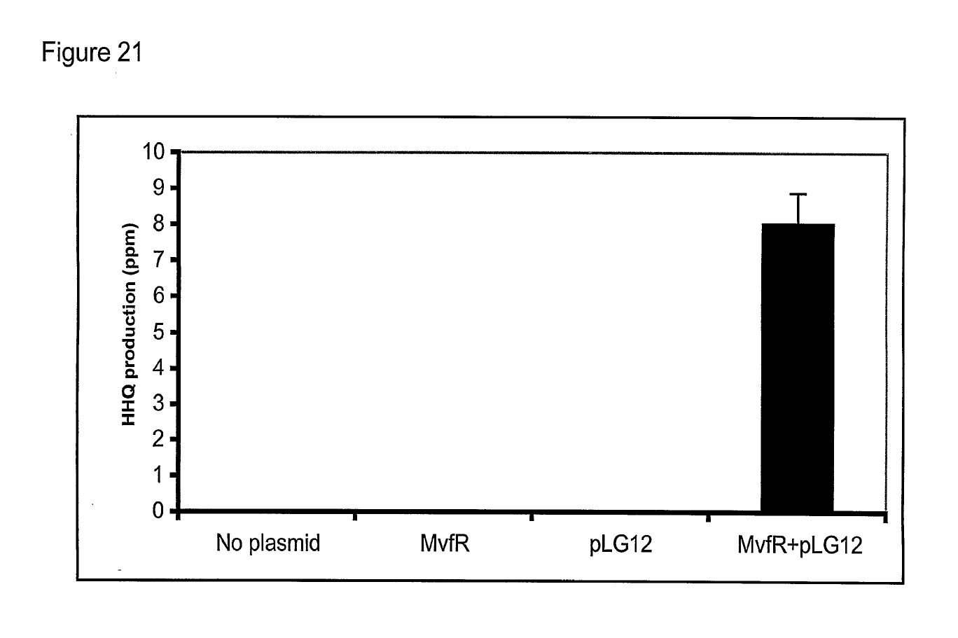

To further confirm the relevance of HHQ to MvfR activity, we developed an HAQ producing heterologous system. E. coli cells containing pDN18-mvfR, which constitutively expresses MvfR, and pLG12, which carries the pqsA-D operon under the control of its own promoter, along with control cells harboring pDN18-mvfR alone, pLG12 alone, or no plasmid, were assayed by LC/MS to measure the four major HAQs in their cell organic extracts. FIG. 21 shows that HHQ and HNQ are only produced in cells that carry both plasmids, demonstrating that MvfR and pqsA-D are sufficient to reconstitute pqs regulation in E. coli (data represented in Table 2).

TABLE 2
HAQ production in E. coli cells containing
pqsA-D and constitutively expressing MvfR.

| E. coli (plasmids) | HHQ (ppb) | HNQ (ppb) | HQNO(ppb) | PQS (ppb) |
|---|---|---|---|---|
| No plasmid | 0.07 | 0.15 | 0 | 0 |
| P_{pqsA}-pqsA-D | 0.1 | 0.2 | 0 | 0 |
| MvfR | 0 | 0 | 0 | 0 |
| MvfR + | 2.17 | 1.35 | 0 | 0 |
| P_{pqsA}-pqsA-D | | | | |

Prevention of the Activation of the Virulence-Related MvfR Transcription Factor Via Compounds that Inhibit the Synthesis of its Ligand.

We have identified inhibitors of the PqsABCD biosynthetic pathway that do not perturb cell growth. These inhibitors disrupt HAQ/PQS synthesis and ultimately limit QS and virulence. Since AA is the precursor of all HAQs, we tested AA analogs for their ability to inhibit this pathway.

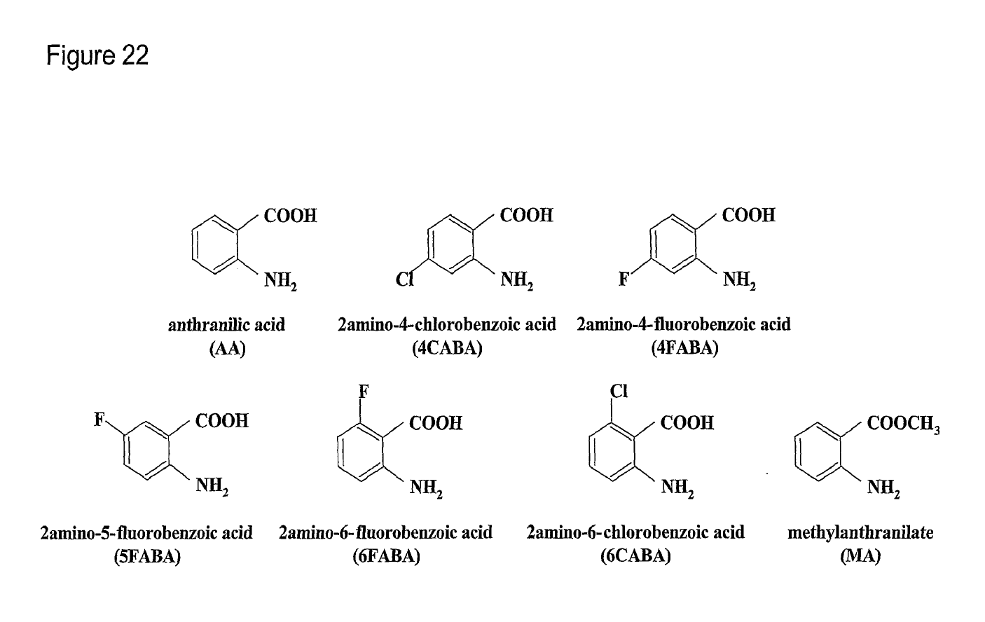

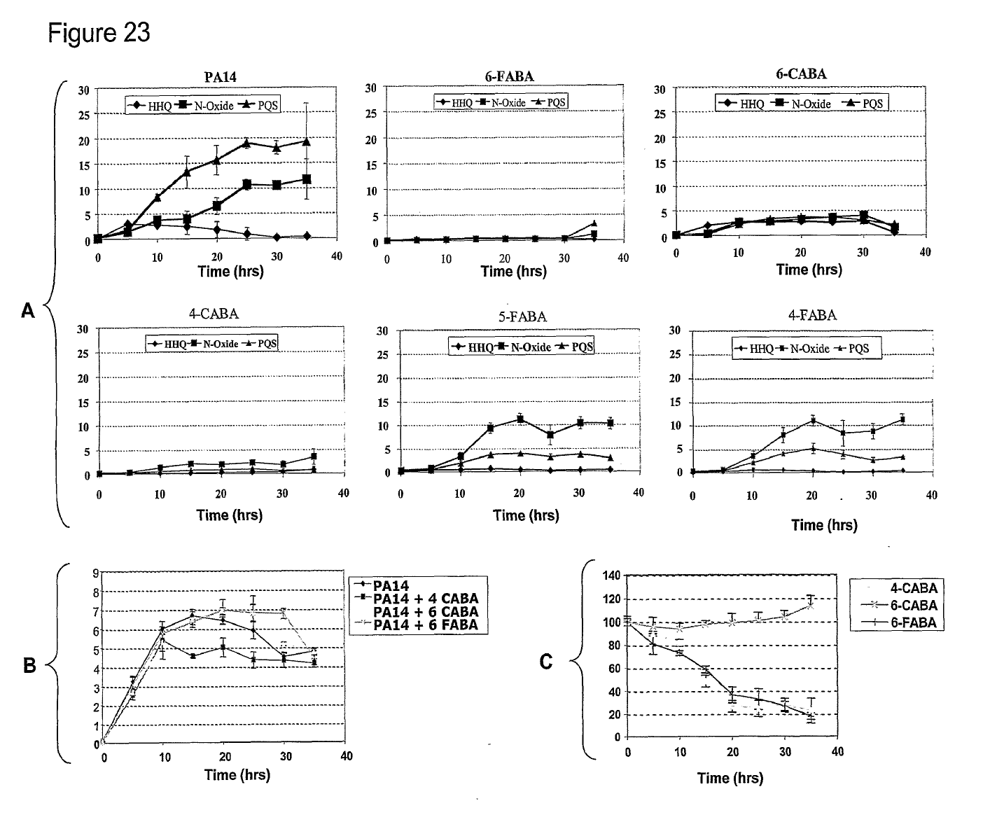

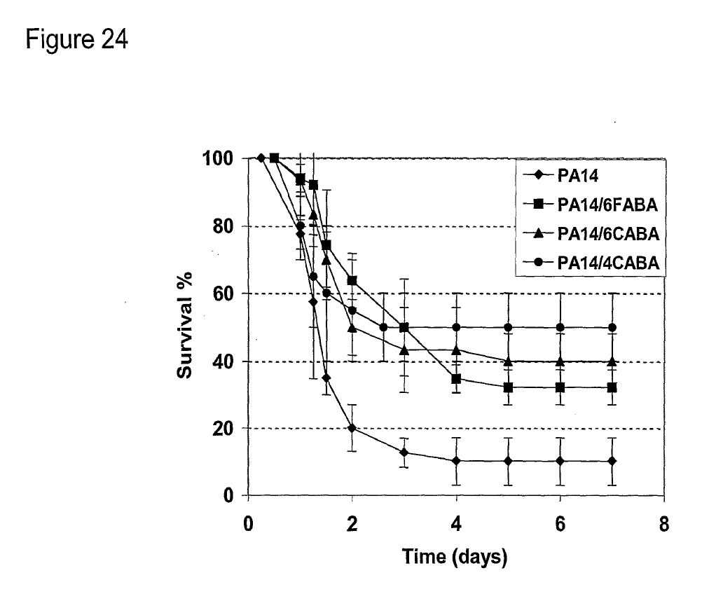

We identified and tested 3 AA analogs, 6-FABA, 5-FABA, and 6-CABA (FIG. 22), which inhibited HAQ production. Specifically, 6-FABA, 6-CABA, and 4-CABA completely halted HAQs synthesis without affecting bacterial growth (FIGS. 23A-23C), and without abrogating pyocyanin production, as would be expected if no PQS was produced. The effectiveness of these compounds demonstrates that AA analogs can specifically block HAQ biosynthesis. Furthermore, we observed that 6-CABA is slowly depleted from bacterial cultures, unlike 6-FABA and 4-CABA which are actively catabolized (FIG. 23C), thus indicating the potential in vivo efficacy of 6-CABA (FIG. 24).

We also tested two related AA analogs, 4-fluoro-2-aminobenzoic acid (4-FABA) and 5-fluoro-2-aminobenzoic acid (5-FABA; FIGS. 23A-23C) for their inhibition of HAQ synthesis and catabolic stability. FIG. 23A demonstrates that only 1.5 mM 6-FABA was required to potently inhibit HAQs. This compound also strongly inhibited production of all principal members of the 3 main families of HAQs: 4-hydroxy-2-heptylquinoline (HHQ), 4-hydroxy-2-heptylquinoline N-oxide (HQNO), and PQS. Importantly, 6-CABA does not inhibit PA14 growth (FIG. 23B), and is stable in P. aeruginosa cultures (FIG. 23C).

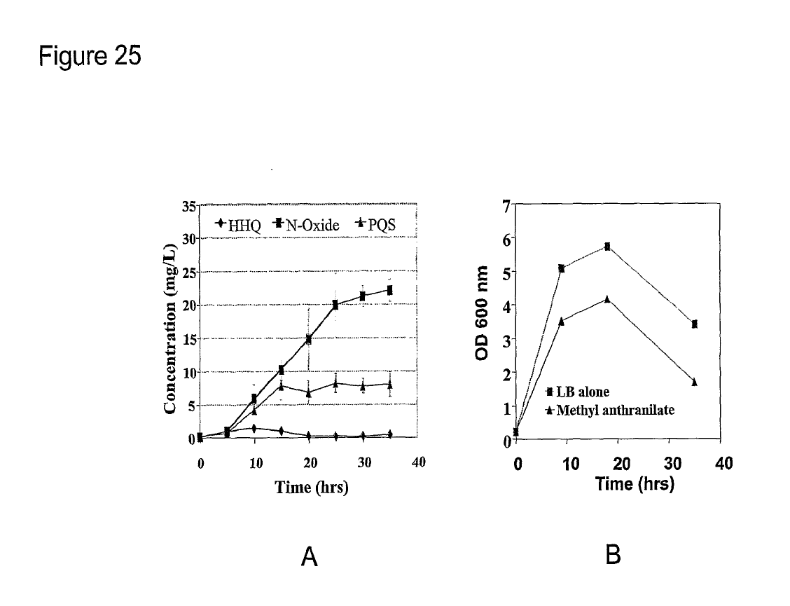

We also tested methylanthranilate (MA) for its inhibitory effect on HAQ/PQS synthesis. Although MA prevents PQS production and reduces the levels of several HAQs, MA produces at least two undesirable side effects: it causes the production of N-oxides at very high concentrations and it inhibits PA14 growth (FIGS. 25A and 25B).

### 6-FABA, 6CABA and 4CABA Limits P. Aeruginosa Virulence in Mice

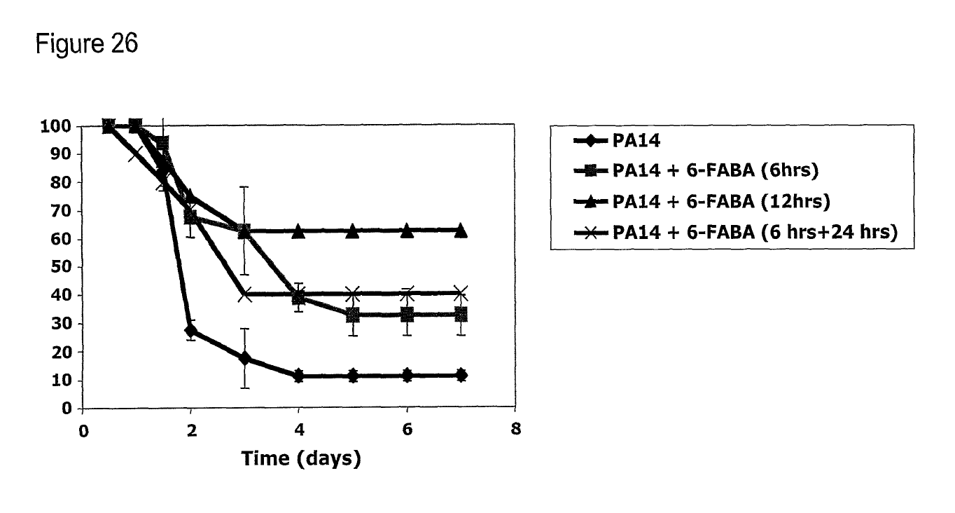

Initial experiments were aimed at defining an appropriate mouse administration protocol for 6-FABA, with regard to concentration, time of administration following infection, and inhibition of virulence. First, following burn, mice were intravenously administered 100 μl 20 mM 6-FABA once or twice in 0.9% NaCl to assess toxicity, with 100 μl being the maximal allowable bolus and 20 mM the maximal 6-FABA solubility. Single injection of 20 mM 6-FABA 6 hrs post-burn is non-toxic, while a significant number of mice die when given a second injection at 12 or 18 hr, and still exhibit 10% mortality with a second injection at 24 hr. Accordingly, burn and PA14 infected (BI) mice were subsequently administered a single injection of 20 mM 6-FABA at 6 or 12 hr post BI; and double injections at 6 and 24 hr BI. FIG. 26 shows that 6-FABA injection 6 hr BI delayed mortality and increased survival following infection with P. aeruginosa from 10% to 40% versus uninjected control DI mice. Animals receiving two injections at 6 and 24 hr post-burn exhibited similar survival rates, but with accelerated mortality. Further protection up to 60% survival was produced by 6-FABA injection 12 hr BI.

We next compared the potential of 6FABA, 6CABA, and 4CABA to reduce P. aeruginosa virulence in a thermal injury mice model. Post burn and PA14 infection, mice received by IV a single injection of 6FABA, 6CABA, or 4CABA at 6 hrs. FIG. 24 shows that 6FABA, 6CABA, and 4CABA increased the final mice survival rate at 7 days post burn and infection by ˜35%, ˜40%, and ˜50%, respectively, as compared to only 10% survival rate in the control infected mice. This demonstrates that, in vivo, all three AA analogs efficiently reduce P. aeruginosa pathogenicity.

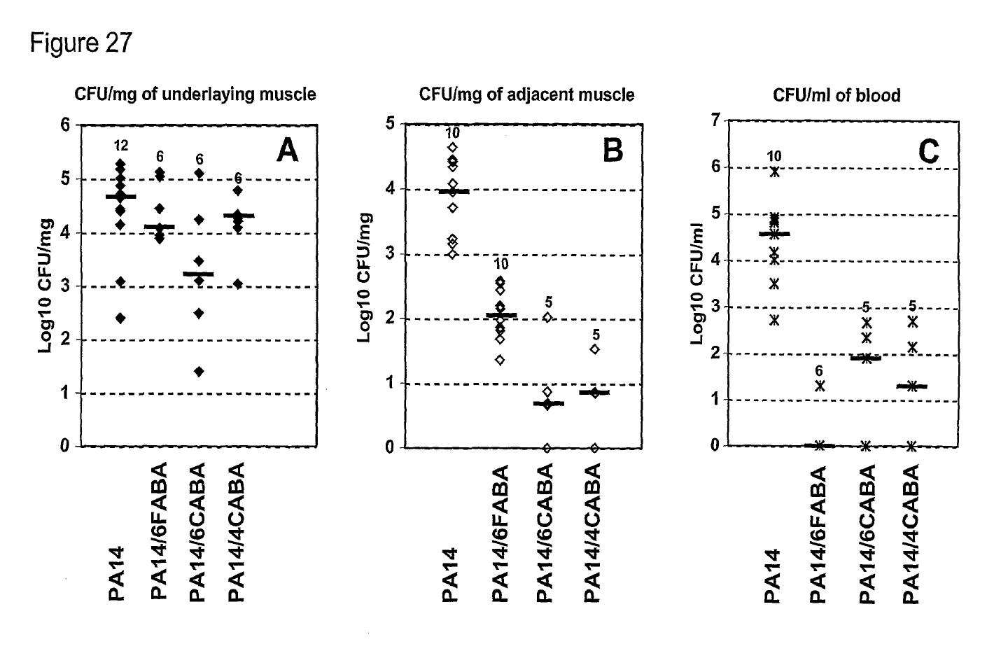

Furthermore, 6-FABA, 6CABA, and 4CABA efficacy in limiting PA14 systemic spread was assessed by comparing burn mice infected with PA14 and then treated with the compounds at 6 hr BI with control untreated BI mice. For each experimental set of n=18, 6 mice were sacrificed at 24 hr, and bacteria numbers were determined in muscle tissue underlaying or adjacent to the 131 site and in blood. The inoculation procedure used in the thermal injury model greatly limits the initial delivery of bacteria to blood and muscle, so bacteria in these tissues reflect post-infection systemic spread. FIG. 27A shows that underlying muscles of animals treated with AA analogs or not treated contain a similar PA14 cfu/mg, demonstrating that AA analogs do not alter bacterial local proliferation. However, the number of PA14 cfu in muscle adjacent to the burn and inoculation site in 6FABA, 6CABA, and 4CABA treated mice is significantly different compared to the control (2-3 log difference), with P values of 0.00001, 0.00066 and 0.0026, respectively (FIG. 27B). The effect of all three compounds in controlling bacterial dissemination throughout the host is further supported by the observation that at 24 hrs blood samples from treated animals harbor at least 2.5 log less PA14 cells than control mice, with P values inferior to 0.005 (FIG. 27C).

The in vitro and in vivo anti-infective efficacy of 6-FABA, 6CABA, and 4CABA demonstrates that AA analogs that inhibit HAQ production restrict P. aeruginosa pathogenesis.

### Example 10

### Treatment of Mice with 6-Fluoro-2-Aminobenzoic Acid in the Burn/Infection Model

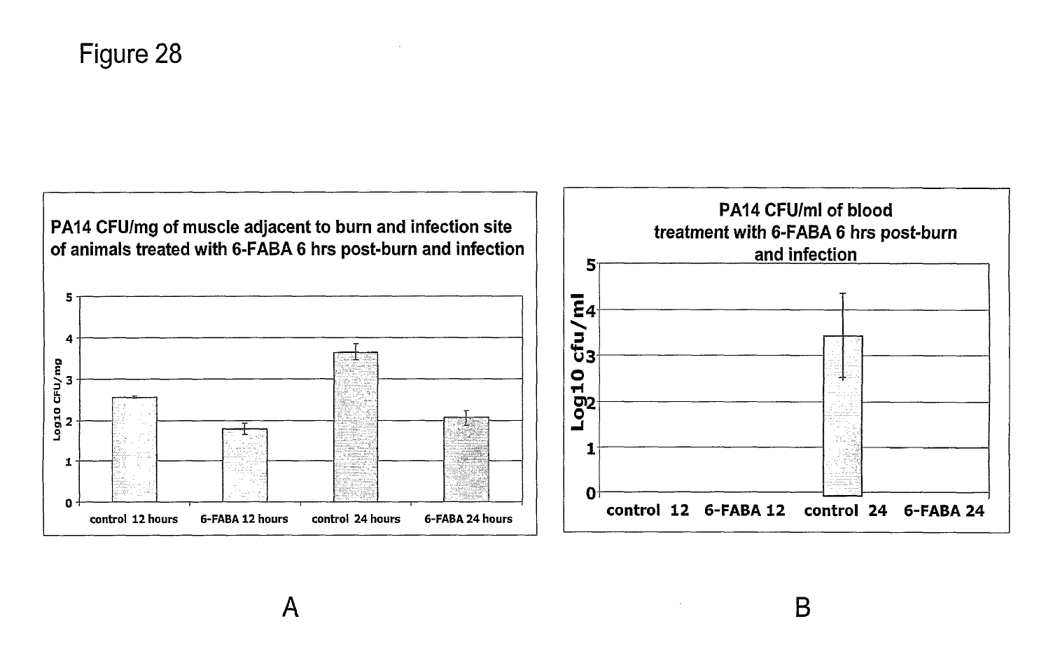

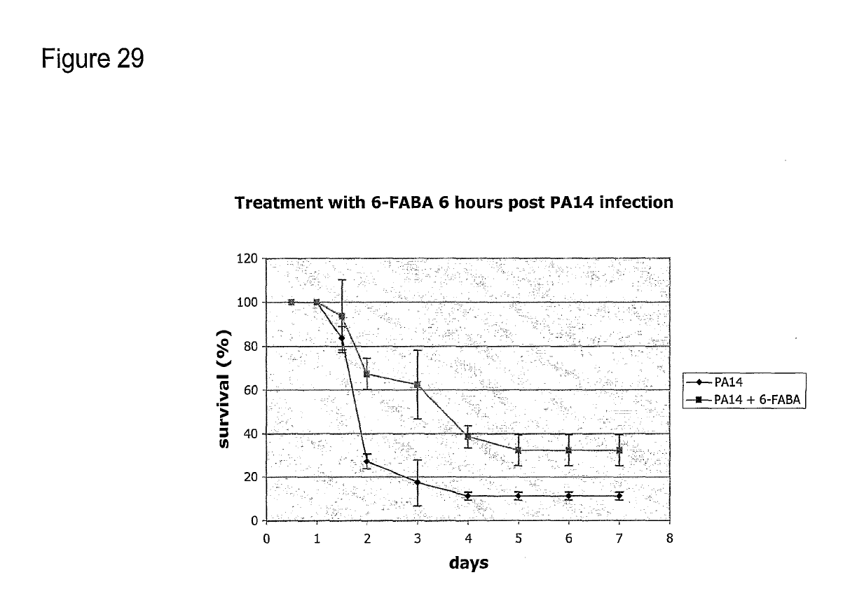

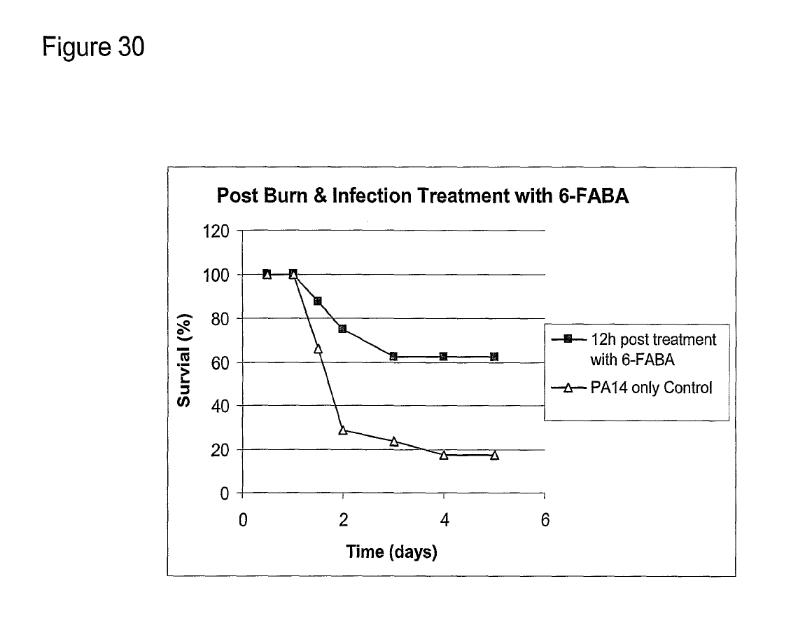

In an experiment to determine the whether 6-fluoro-2-aminobenzoic acid (6FABA) affects the systemic spread of P. aeruginosa cells in a mouse burn/infection model, the results of which are shown in FIGS. 28A and 28B, 15 mice were treated with 100 μL of a 20 mM solution of 6-FABA 6 hrs or 12 hours post-burn and infection, and the PA14 cfu/mg of tissue (i.e., in blood and in muscle adjacent to the burn and infection) was determined at 24 hours and 36 hours post-burn. FIG. 28A shows that 6-FABA limits significantly the systemic dissemination of P. aeruginosa. A dramatic effect is seen in the blood samples, as no bacteria are detected in the blood of treated mice at 24 hrs (FIG. 28B). These results correlate with the increased survival of the mice, as shown in FIGS. 29 and 30, for 6 hour and 12 hour post injury treatments, respectively.

### Example 11

### The Antranilic Acid (AA) Analogs 6-CABA and 6-FABA Act Through Inhibition of the PqsA Enzymatic Activity

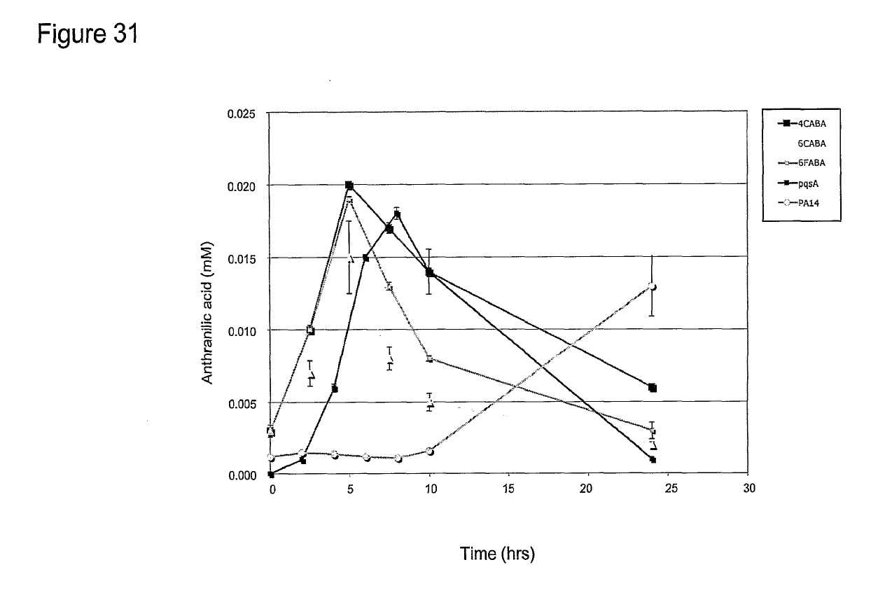

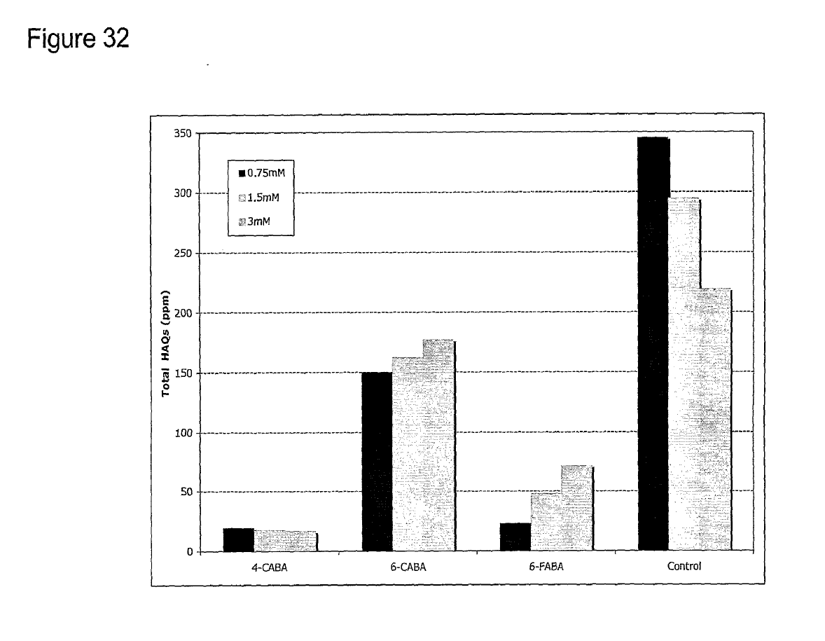

In P. aeruginosa anthranilic acid is also produced by the enzymatic systems PhnAB or TrpEG. Upon addition of 6-CABA, 6-FABA, or 4-CABA, we observed a transient accumulation of anthranilic acid, contrary to the control (FIG. 31). In addition a pqsA mutant also showed a transient accumulation of anthranilic acid very similar to the one observed with these inhibitors. This accumulation could be due to inhibition of PqsA or of any enzyme downstream in the HAQ biosynthesis. However, the addition of increasing concentrations of AA in culture containing 1.5 mM of any of these inhibitors reversed the inhibition (FIG. 32) and lead to an increase in HAQ production, at least for 6-FABA and 6-CABA. This reversal could only be explained by a competitive inhibition of an enzyme acting on AA. Because pqsA is the only enzyme of the pqsA-E operon to present similarities with enzymes involved in the activation of aromatic acid group, like the one found in anthranilic acid, we conclude that these inhibitors act on PqsA in a competitive manner with its substrate AA.

### Example 12

### Identification of Inhibitors that Block the MvfR Ligand Binding Site

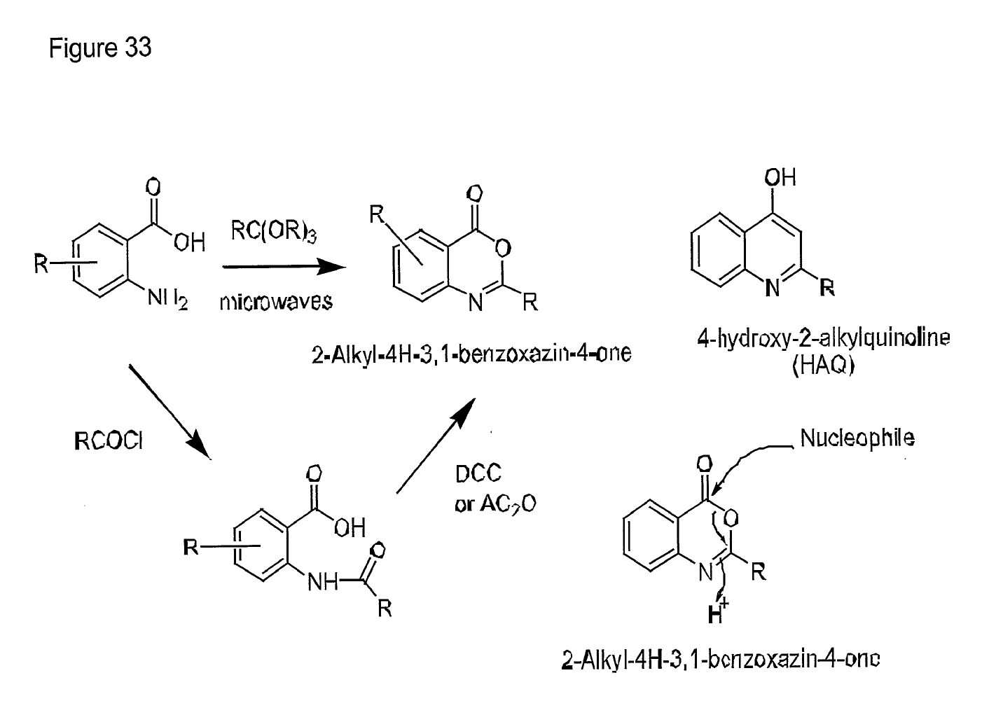

Compounds that block the MvfR ligand binding site may also have beneficial immunomodulatory activity, and therefore may be used to prime host defenses to prevent or limit bacterial viability. Such compounds should be structurally similar to PQS, and preferably be able to chemically modify a reactive group located on the tryptophan residue in the ligand-binding site. Such compounds include a series of 2-alkyl-4H-3,1-benzoxazin-4-ones. These compounds, which are structurally related to HAQs (see FIG. 33), bear a reactive carbonyl group susceptible to nucleophilic attack. Such molecules can acylate residues on chymotrypsin and can be readily synthesized with alkyl chains of different lengths from anthranilic acid (AA) plus an orthoester using microwave irradiation (Khajavi et al., J. Chem. Res. 8:286-287, 1997), or via activation of an intermediate N-acetylanthranilic acid product of AA reacted with an acid chlorine or anhydride (Ossman and Barakat, Saudi Pharm. J. 2:21-31, 1994). Substituted AA analogs bearing an ortho or para electron withdrawing (R═Cl, F) or donating groups (R═CH3 or OCH3) can also be used as starting material for the synthesis of modified benzoxazinones in order to modulate the reactivity of the electrophilic carbonyl of the benzoxazinone.

2-heptyl-4H-3,1-benzoxazin-4-one can be synthesized by reacting AA with heptanoyl chloride, and treating the resulting amide with acetic anhydride (Ossman and Barakat, supra). To confirm that alkyl-benzoxazinone covalently binds the ligand binding site, we can add it to purified MvfR, and determine by MS that the MvfR mass increases by the equivalent mass of one inhibitor molecule. Furthermore, we can verify that the compound reacts at the defined binding site, and blocks ligand binding: the inhibitor-MvfR complex can be digested with trypsin and the peptide covalently bound to the compound can be analyzed by LC/MS; and following exposure to the inhibitor, we can test whether binding is selectively displaced by non-radioactive ligand by assessing if column-immobilized MvfR-TAP tagged protein no longer binds radiolabeled ligand.

If 2-heptyl-4H-3,1-benzoxazin-4-one, or other tested analogs, inhibit ligand binding and covalently bind MvfR, we can partially trypsin digest the inactivated MvfR and analyze the tryptic fragments by nano LC/MS. Chromatographic separation of the peptides allows detection of the chemically modified peptide; and disappearance of the underivatized peptide and corresponding appearance of a peptide with a mass increment equivalent to that of the inhibitor provides the mass of the unbound peptide, and thus identifies the general covalent attachment site. We have previously performed a similar analysis on a paraben degrading esterase following the addition of an irreversible inhibitor (Valkova et. al., J. Biol. Chem. 278:12779-12785, 2003).

If alkylbenzoxazinones inhibit ligand binding to MvfR in vitro, we can evaluate whether their addition to PA14 cells inhibits pqsA-lacZ expression in vivo. Inhibitory activity can be confirmed by assaying for reduced pyocyanin and HAQ production, which are under MvfR control.

### Example 13

### Identification of Mouse Genes Induced in Response to 2AA Treatment

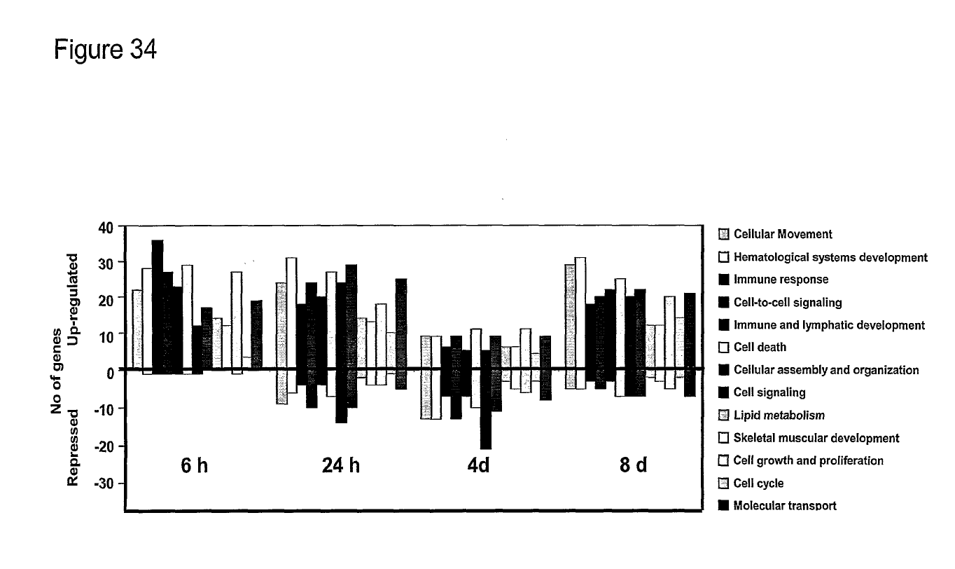

To identify how 2AA pre-exposure promotes host defense to better protect against P. aeruginosa infection, we carried out whole genome gene expression studies of mice for several experimental conditions using the mouse full-thickness skin thermal injury infection model, including naïve; BI alone; 2AA treatment alone; 2AA pretreatment plus BI. The mice were injected IP, as we were not yet aware of the improved benefit obtained with IV delivery. Whole blood was collected after 0, 6, 24, 96 and 192 hr post 2AA injection. Also, pretreated BI mice were exposed to 2AA at 1 and 4 days pre-BI, with blood samples collected 24 hr post-BI. The transcriptome profiles were then determined and compared, and genes with statistically significant differences in their expression levels between the samples were further analyzed by pair-wise comparison (see experimental design, section 2.2). These preliminary studies collectively show that 835 genes have >2× expression differences at least at one point in the mice treated with 2AA versus the control mice. FIG. 34 shows a functional categorization of these genes, which include chemotaxis, immune response, cell-cell signaling, and hematological systems development; plus cell cycle, cell proliferation, cell death, and molecular transport. Both up- and down-regulated genes are observed. The majority of the up-regulated genes are seen within 6 hr of 2AA injection, and peak at 24 hr.

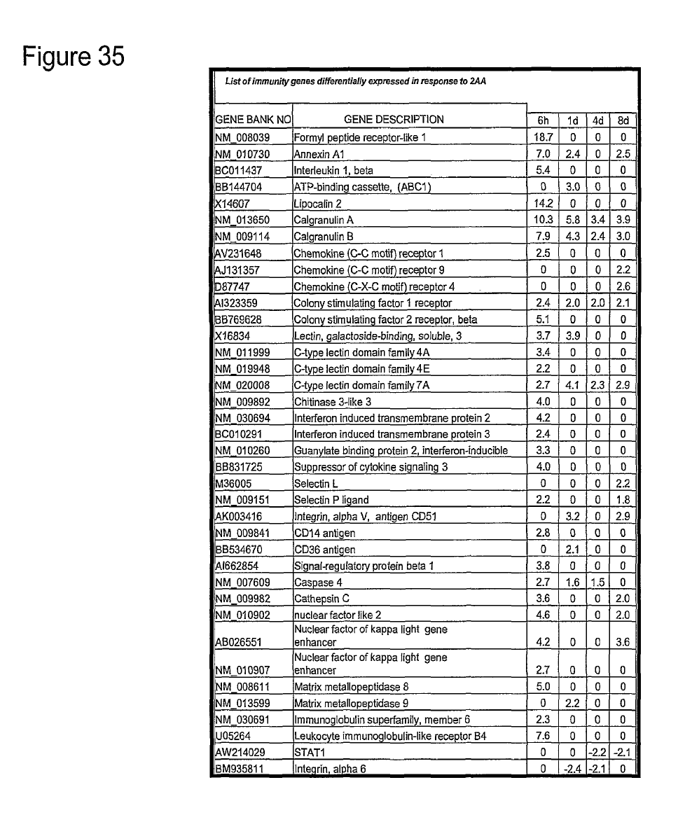

FIG. 35 shows Table 3, which lists the 2AA differentially responsive genes that encode immune response functions. For instance, after 6 hr 2AA treatment, IL-1β is up-regulated together with the ABC transporter needed for secretion of mature IL-1β. Other up-regulated innate immunity genes include the formyl peptide receptor like gene and Lipocalin 2. Lipocalin 2 knockout mice are more susceptible to E. coli infection. 2AA also stimulates chemotaxis (chemokines and CSFs) antimicrobial response (C-type lectins, chitinases), and nuclear factor functions.

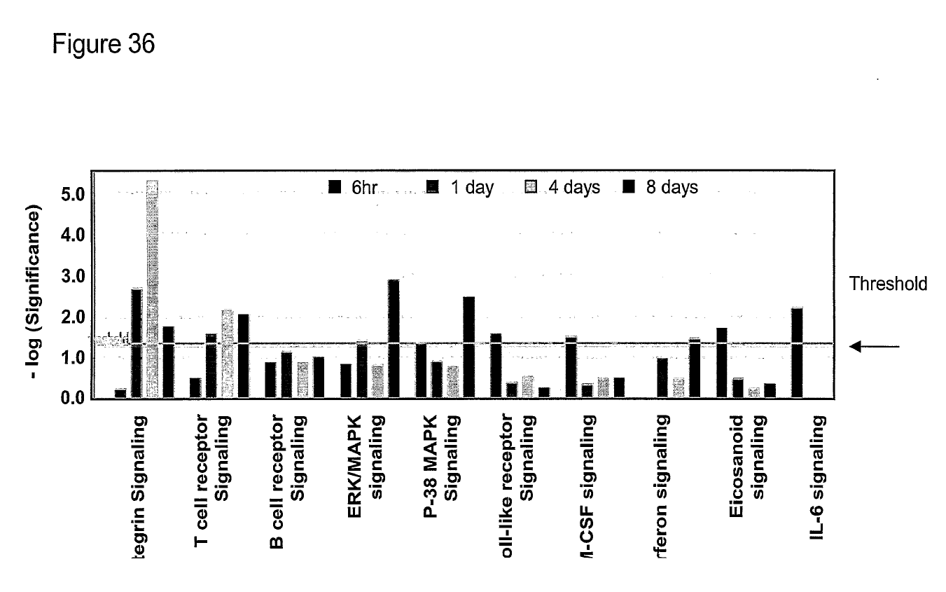

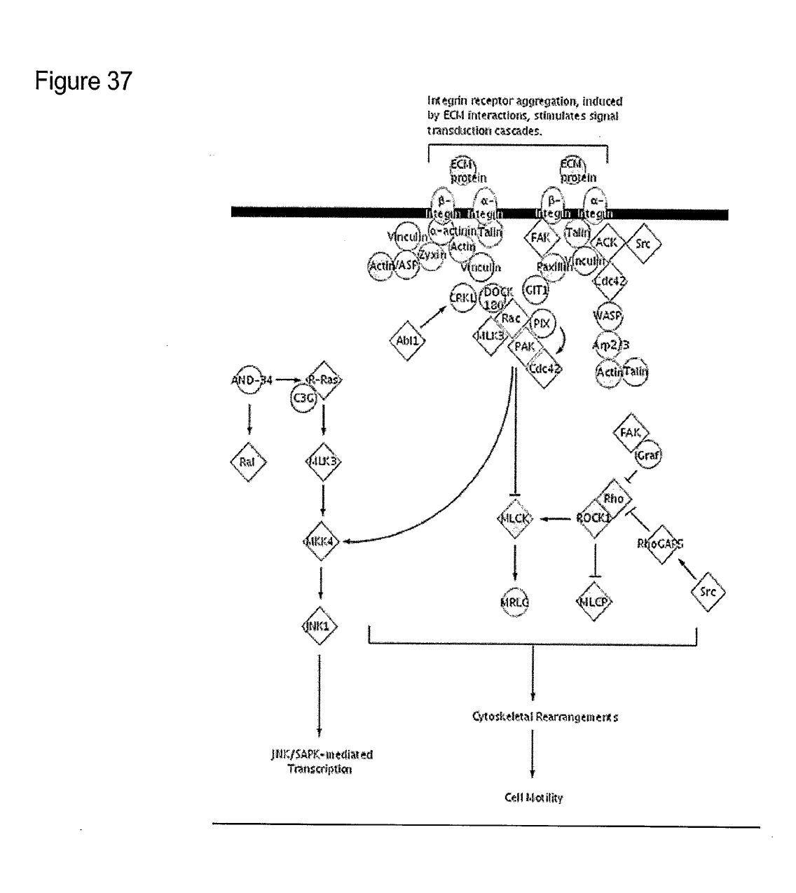

We used the Ingenuity pathway analysis (IPA) program to identify signaling pathways with altered expression at 96 hr post-2AA treatment. As shown in FIG. 36, the most significant change was observed in the intergin signaling pathway at 96 hours. Multiple genes were found to be down-regulated in the pathway. A cartoon of the pathway depicting the changes in the signaling component are presented in FIG. 37. These components control cell motility, and the modulation in their expression suggests that 4 days after 2AA treatment cell motility is reduced, which is likely responsible for the recruitment of innate immune effecter cell at the site of infection. The T-cell receptor signaling was the next most significant pathway affected. Three positive regulators of the T-cell receptor signaling pathway (MEK, PP3C, and RASGRP) were induced at 24 hrs and stayed induced up to 8 days with peak expression at 4 days. Three genes were also found to be down-regulated, two of which are negative regulators (Lymphocyte specific protein and Calmodulin3) of T-cell activation. These results indicate that T-cell mediated immune response may peak at 4 days and may have a role in protecting the host.

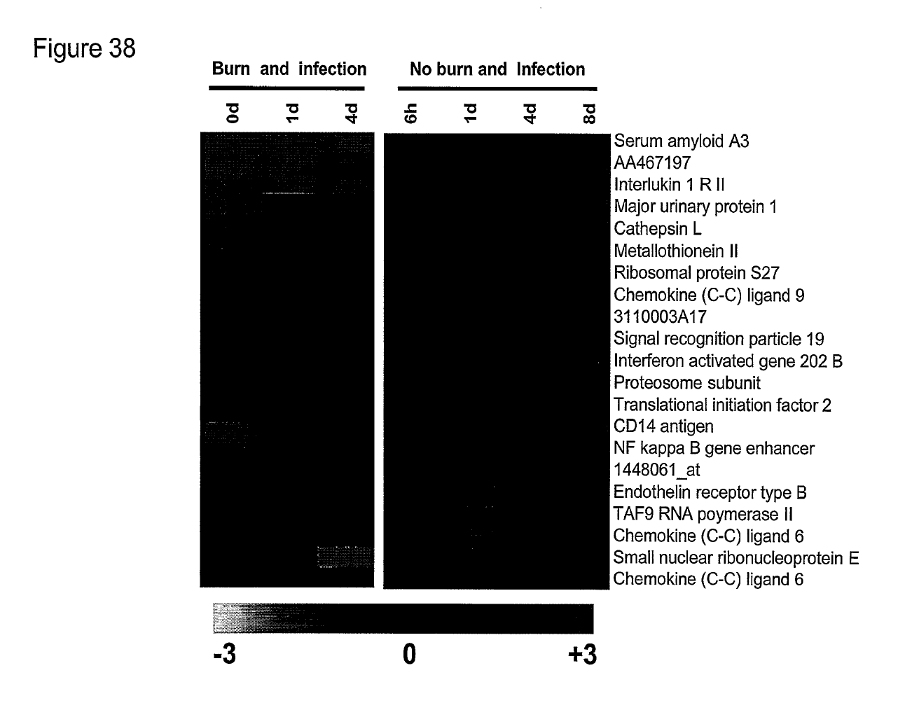

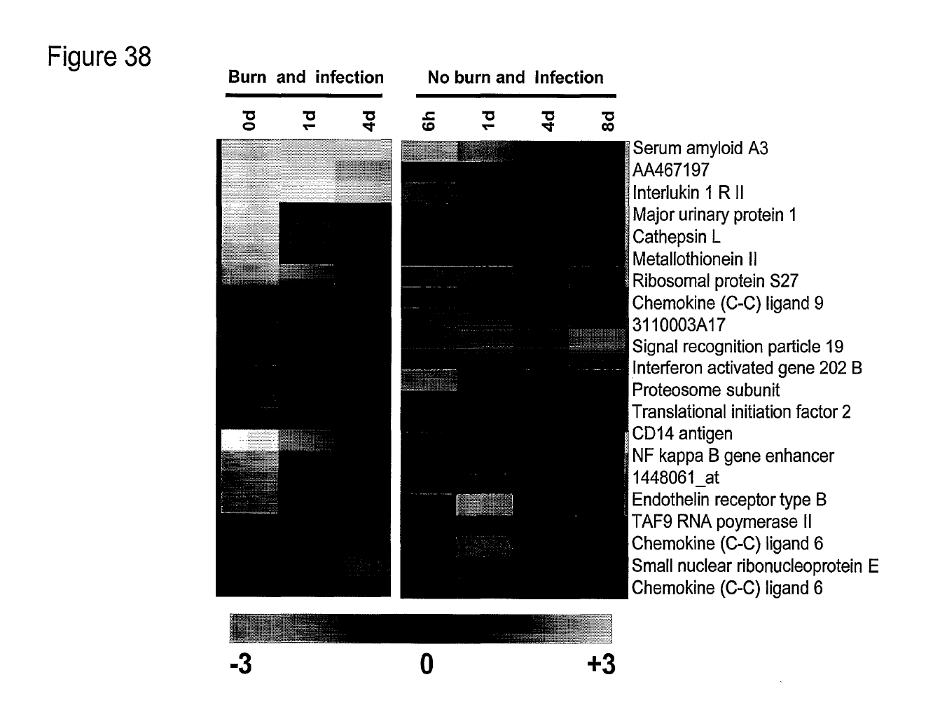

Consistent with the down-regulation of the cytoskeleton proteins, ERK signaling components that negatively regulate the cytoskeleton reorganization were found to be up-regulated. However, we found 7/8 genes involved in the P38 MAPK pathway to be induced, which suggests an increase in P38 signaling. These genes include moderate activation of TGFβ receptors I and II suggesting secretion of TGFβ by the activated T-cells. TGFβ is known to negatively regulate inflammation. Since T-cell mediated responses are important for adaptive immunity, these changes may explain the long-term protection obtained with 2AA. B-cell activation may also be important at later time points. In summary, these data suggest 2AA provides protection to bacterial infection by significantly altering the expression of several immune signaling pathways that mediate various immune response functions. The analyses of the 2AA-treated BI samples surprisingly do not identify any up-regulated, and only 23 down-regulated, genes. These latter genes function in the inflammatory response that is highly induced by BI (FIG. 38, left panel), and are dramatically reduced in the 2AA treated samples. Interestingly, these genes are either unchanged, or induced by 2AA alone (FIG. 38, right panel). These results indicate that burn and P. aeruginosa infection leads to an exaggerated host inflammatory response; and 2AA is able to turn down this response, which in turn likely contributes to enhanced host survival to BI.

### Example 14

### Genome Expression Studies of Mice after 2′-Aminoacetophenone Treatment

In experiments to determine if T-aminoacetophenone acts as an immunomodulator, eight 6-week old CD-1 male mice were treated intraperitonialy with 500 μl of a 20 mM solution of T-aminoacetophenone in 0.9% NaCl (aq). A control group of mice was administered only 0.9% NaCl. Blood was withdrawn from the mice at time points of 6 hours, 24 hours, 96 hours, and 192 hours, and RNA was extracted from each blood sample.

Mouse GeneChip™ arrays, manufactured by Affymetrix (Santa Clara, Calif.), were used to compare the profile of gene expression in the blood samples of mice. The biotin-labeled cRNA used to hybridize to the GeneChips was prepared from the RNA extracted from the blood samples according to the Affymetrix GeneChip Expression Analysis Technical Manual (Research Genetics, Huntsville, Ala.).

The expression profiles from treated and untreated mice were compared and an additional constraint of a minimum expression ratio of 1 was applied to control false positives to 5%. The gene list was clustered and the genes showing upregulation at any of the time points considered were selected. Table 3 shows some representative genes activated after 2′-aminoacetophenone treatment. Most of the genes listed in the table function in immune response or host-pathogen interaction. The table includes several cytokines and their receptors, genes involved in immune cell proliferation, and calcium-binding proteins that have a role in downstream signaling.

TABLE 3

| Mouse Gene | | Fold Change | | | |
|---|---|---|---|---|---|
| Bank No. | Gene Description | 6 h | 24 h | 96 h | 192 h |

Cytokines and activated proteins

| | | | | | |
|---|---|---|---|---|---|
| NM_011940 | Interferon-activatable protein (Ifi202) | 29.8 | 9.4 | 9.4 | 9.1 |
| AV229143 | Interferon activated gene (202A) | 21.2 | 10.9 | 6.8 | 6.2 |
| BB193024 | Interferon-induced transmembrane protein-2 | 5.0 | 1.0 | 0.1 | 0.3 |
| NM_030694 | Interferon-induced transmembrane protein-3 | 4.7 | 1.1 | 0.2 | 0.6 |
| BC010291 | Interferon-inducible protein (Cd225) | 1.3 | 0.0 | −0.3 | −0.3 |
| BC012653 | Chemokine (C-X3-C) receptor 1 | 30.6 | 11.6 | 0.0 | 0.0 |
| BC011092 | Chemokine (C-C) receptor 1 | 1.2 | −0.9 | −0.7 | −0.3 |
| BC011437 | IL-1β | 3.5 | 0.6 | 0.2 | 0.6 |
| BE285634 | Interleukin-1 receptor accessory protein | 1.0 | 0.2 | −1.1 | −0.2 |
| NM_009883 | Nuclear factor induced by IL-6 (NF-IL6) | 1.0 | 0.3 | −0.1 | −0.1 |
| AI462015 | Nuclear factor of kappa light chain gene | 2.3 | 0.4 | 0.4 | 0.7 |
| | enhancer in B-cells inhibitor, alpha | | | | |
| BC021916 | EMAPI, cytokine activity | 1.7 | 0.3 | −0.3 | −0.2 |
| BB831725 | Suppressor of cytokine signaling 3 | 20.8 | 6.5 | 2.0 | 5.9 |
| BB241535 | Suppressor of cytokine signaling 3 | 4.9 | 1.1 | 0.3 | 1.0 |

Innate immunity components

| | | | | | |
|---|---|---|---|---|---|
| NM_008879 | Pls2, L-fimbrin, calcium ion binding | 20.4 | 8.4 | 0.0 | 0.0 |
| | lymphocyte cytosolic proteing 1 (Lep 1) | | | | |
| NM_011355 | PU.1, transcription factor complex cell growth | 17.0 | 0.0 | 9.2 | 6.0 |
| | and/or maintenance | | | | |
| AV026617 | FBJ osteosarcoma oncogene | 15.0 | 23.8 | 0.0 | 10.5 |
| BB769628 | Colony stimulating factor 2 receptor, beta 1 | 14.9 | 6.9 | 3.2 | 5.2 |
| NM_011315 | Saa-3 lipid transported activity acute phase | 4.7 | 5.6 | 1.5 | −0.2 |
| | response | | | | |
| AI323359 | Csfmr, colony stimulating factor IR, regulator | 2.3 | 0.7 | 0.0 | 0.3 |
| | of myeloid cell proliferation | | | | |
| AF220015 | Rpt-1; regulatory protein, T lymphocyte 1 | 2.3 | 0.6 | 0.5 | 0.6 |
| BC022943 | Lymphocyte cytosolic protein 1 | 2.0 | 0.2 | −0.4 | −0.1 |
| AV318494 | Myeloid cell leukemia sequence 1 | 1.8 | 1.1 | 0.3 | 1.2 |
| BC003839 | Myeloid cell leukemia sequence 1 (Mcl1) | 1.5 | 0.0 | −0.4 | 0.1 |
| U72644 | Leukocyte specific transcript 1 | 1.6 | 0.9 | −0.2 | 0.1 |
| NM_008677 | p40phox; intracellular signaling cascade | 1.1 | 0.2 | 0.0 | 0.1 |
| | neutrophil cytosolic factor 4 (Ncf4) | | | | |

Calcium binding or channels

| | | | | | |
|---|---|---|---|---|---|
| NM_011999 | DCIR; C-type (calcium dependent, | 22.8 | 8.0 | 5.3 | 9.6 |
| | carbohydrate recognition domain) lectin | | | | |
| X14607 | Lipocalin 2 | 13.9 | 0.2 | 0.2 | 0.2 |
| AI648846 | Solute carrier family 6 | 10.1 | 8.0 | 3.7 | 7.8 |
| NM_011313 | Cacy; calcium binding protein A6 | 5.7 | 2.3 | 0.5 | 1.4 |
| | (calcyclin)cyclin-dependent protein kinase | | | | |

Receptors

| | | | | | |
|---|---|---|---|---|---|
| NM_030691 | Integral to membrane immunoglobulin | 14.9 | 3.5 | 0.0 | 5.6 |
| | superfamily, member 6 (Igsf6) | | | | |
| NM_008039 | Receptor activity [formyl peptide receptor, | 13.6 | 0.6 | 0.2 | 0.2 |
| | related sequence 2] (Fpr-rs2) | | | | |
| BB784999 | Triggering receptor expressed on myeloid cells 1 | 11.6 | 5.9 | 4.6 | 0.0 |
| U05264 | Glycoprotein 49B (Gp49b) | 4.7 | 1.0 | 0.6 | 0.9 |
| AF237910 | Membrane-spanning 4-domains, subfamily A | 2.8 | 0.5 | 0.0 | 0.5 |
| NM_011087 | Paired-Ig-like receptor A1 (Piral) | 1.9 | 0.9 | 0.2 | 0.1 |

A more stringent analysis by applying a higher cutoff revealed 10 additional genes that are revealed to be upregulated 6 hours after 2′-aminoacetophenone. These genes are shown in Table 4. As before, some of these genes are involved in the perception and transduction of the immune response.

TABLE 4

| Mouse Gene | Human | |
|---|---|---|
| Bank No. | homolog | Gene Description |
| NM_013650 | P49006 | Calgranulin A |
| NM_008039 | B42009 | FPRL1, formyl peptide receptor-like 1 |
| BC027285 | NP_066362.1 | IFITM1 |
| AF099975 | None | Schlafen 4 |
| X14607 | P80188 | Lipocalin 2 |
| NM_009114 | P06702 | Calgranulin B |
| NM_009892 | NP_068569.1 | Chitinase 3-like 1 and 2, no 3 |
| AV110584 | P49006 | MARCKS-like protein |
| AA666504 | Q14508 | WDNM1 |
| BB769628 | P32927 | Colony stimulating factor receptor 2 β |

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1-14. (canceled)

15. A method for enhancing the innate immune response for mitigating the effects or propagation of a disease in an asymptomatic animal, said method comprising administering to said animal an effective amount a compound having the formula: 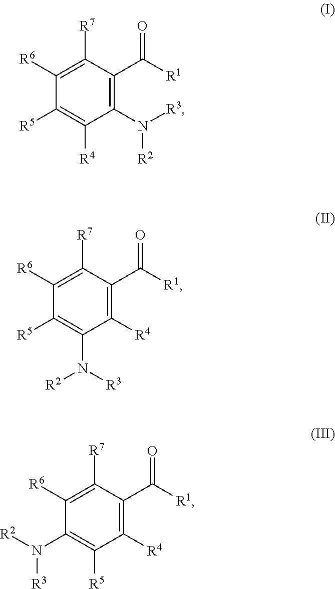
or a pharmaceutically acceptable salt or prodrug thereof, wherein
R¹ is optionally substituted C₁₋₁₂ alkyl, optionally substituted C₃₋₈ cycloalkyl, optionally substituted C₂₋₁₂ alkenyl, optionally substituted C₂₋₁₂ alkynyl, optionally substituted C₁₋₄ alkaryl, or optionally substituted C₁₋₄ alkheterocyclyl;
each of R² and R³ is, independently, H, optionally substituted C₁₋₆ alkyl, optionally substituted C₁₋₄ alkaryl, or optionally substituted C₁₋₄ alkheterocyclyl, or R², R³, and the nitrogen to which they are bonded together form a nitro group;
R⁴ is H, Hal, OH, or C₁₋₆ alkoxy; and
each of R⁵, R⁶, or R⁷ is, independently, H, OH, Hal, optionally substituted C₁₋₆ alkyl, or optionally substituted C₁₋₆ alkoxy.

16. The method of claim 15, wherein said compound is 2′-aminoacetophenone.

17. The method of claim 15, wherein said compound is 2′-amino-3-hydroxyacetophenone.

18. The method of claim 15, wherein said disease is a bacterial infection, a viral infection, an autoimmune disease, an allergic condition, or cancer.

19. The method of claim 18, wherein said bacterial infection is the result of a Gram-negative bacterium.

20. The method of claim 18, wherein said bacterial infection is the result of Vibrio harveyi, Vibrio cholerae, Vibrio parahaemolyticus, Vibrio alginolyticus, Pseudomonas phosphoreum, Pseudomonas aeruginosa Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes, or Staphylococcus aureus.

21. A method for enhancing the innate immune response for mitigating the effects or propagation of a disease in an asymptomatic animal, said method comprising administering to said animal an effective amount a compound selected from the group consisting of:

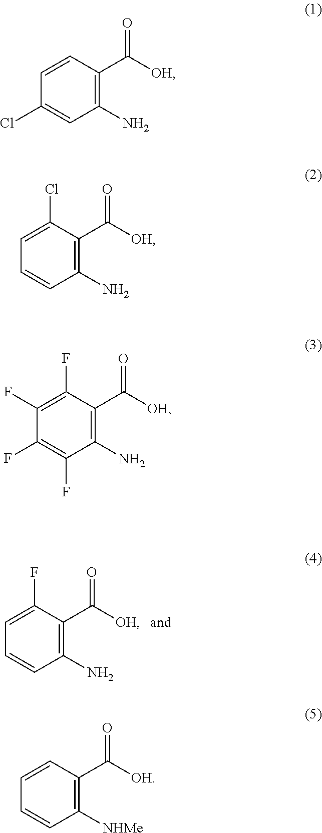 [SMILES: Nc1cc(Cl)ccc1C(=O)O ; Nc1cccc(Cl)c1C(=O)O ; Nc1c(F)c(F)c(F)c(F)c1C(=O)O ; Nc1cccc(F)c1C(=O)O ; CNc1ccccc1C(=O)O]

22. The method of claim 21, wherein said disease is the result of a bacterial infection, fungal infection, or viral infection.

23. The method of claim 22, wherein said bacterial infection is the result of a Gram-negative bacterium.

24. The method of claim 21, wherein said disease results from Vibrio harveyi, Vibrio cholerae, Vibrio parahaemolyticus, Vibrio alginolyticus, Pseudomonas phosphoreum, Pseudomonas aeruginosa Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes, or Staphylococcus aureus.

25. The method of claim 21, wherein said compound does not affect the viability of bacteria, fungus, or virus responsible for said infection.

26-36. (canceled)
